(19) United States
(12) Patent Application Publication
(10) Pub. No.: US 2022/0098683 A1
(43) Pub. Date: Mar. 31, 2022

(54) METHODS AND COMPOSITIONS FOR DETECTION OF HPV DNA AND DIAGNOSIS AND MONITORING OF HPV-ASSOCIATED CANCERS

(71) Applicant: MEMORIAL SLOAN KETTERING CANCER CENTER, New York, NY (US)

(72) Inventors: Daniel Smith Higginson, Tenafly, NJ (US); Rama Rao Damerla, Manipal (IN)

(21) Appl. No.: 17/196,631

(22) PCT Filed: Sep. 10, 2019
(86) PCT No.: PCT/US2019/050365

§ 371 Date: Mar. 9, 2021

Related U.S. Application Data
(60) Provisional application No. 62/729,321, filed on Sep. 10, 2018.

(51) Int. Cl. C12Q 1/70; C12Q 1/6886

(52) U.S. Cl. CPC C12Q 1/708; C12Q 1/6886; C12Q 2600/118; C12Q 2600/112; C12Q 2600/106

(57) ABSTRACT

The present invention provides methods and compositions for the detection of HPV DNA in patient samples with high sensitivity and specificity. Such methods are useful in screening for HPV-associated malignancies. In some embodiments the present invention provides methods and compositions for the detection of HPV16, HPV33 and/or HP VI 8 DNA. In some embodiments the present invention provides methods and compositions for the detection of HPV DNA from squamous cell carcinomas, such as oropharyngeal squamous cell carcinomas and anal squamous cell carcinomas.

### CROSS-REFERENCE TO RELATED APPLICATIONS

This application claims the benefit of priority of U.S. Provisional Patent Application No. 62/729,321 filed on Sep. 10, 2018, the content of which is hereby incorporated by reference in its entirety.

### STATEMENT REGARDING FEDERALLY SPONSORED RESEARCH

This invention was made with government support under grant number CA008748 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 10, 2019, is named MSKCC_032_WO1_SL.txt and is 21,089 bytes in size.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Numbers in superscript or parentheses following text herein refer to the numbered references identified in the “Reference List” section of this patent application.

### BACKGROUND

Cancers driven by human papillomaviruses (HPV) include squamous cell carcinomas of the oropharynx, cervix, vulva, vagina, anal canal and penis. While Papanicolaou (Pap) smears are widely used for early detection of HPV-associated lesions in the cervix, effective screening approaches for other HPV-associated cancers are lacking. For example, HPV-associated oropharyngeal cancers (HPV+OPSCCs) represent a large cohort of HPV-associated cancers for which there is currently no effective screening paradigm. Thus, accurate biomarkers for diagnosis and monitoring of a variety of HPV-associated cancers are urgently needed.

Circulating, tumor-derived DNA (ctDNA) is an emerging biomarker category. Detection of virally-derived ctDNA has several advantages as compared to detection of other non-virally-derived ctDNAs. For example, because the substrate for detection is viral rather than host, the signal to noise ratio is improved. Similarly, because there are often multiple copies of viral DNA within one cancer cell, the amount of virally-derived ctDNA in the blood is often much higher than that of other ctDNAs. ctDNA detection of tumor derived Epstein-Barr Virus (EBV) DNA has been successful for early detection of nasopharyngeal cancer (NPC)⁴ and persistent EBV ctDNA levels are a negative predictive factor for recurrence following chemoradiation.⁵⁻⁸ However, to date, detection of HPV plasma ctDNA has shown only modest sensitivity in patients with gross disease (19-65%).⁹⁻¹² As such, there is a need in the art for new and improved methods detecting and measuring circulating HPV ctDNA.

### SUMMARY OF THE INVENTION

Some of the main aspects of the present invention are summarized below. Additional aspects are described in the Detailed Description of the Invention, Examples, Drawings, and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention.

The present invention is based, in part, on a series of important developments and discoveries that are described in more detail in the “Examples” section of this patent specification—which describes the successful development of new and improved compositions and methods that enable HPV DNA (such as ctDNA) to be detected and measured with high sensitivity and specificity in various HPV-associated tumors and in various clinical settings. Such compositions and methods are particularly useful for screening for HPV-associated malignancies. Using the compositions and methods provided herein, near universal detection of several HPV-associated cancers was achieved. Furthermore, HPV DNA detected using these compositions and methods exhibited a quantitative relationship to tumor volume-enabling the kinetics of such HPV DNAs (ctDNAs) to be accurately tracked over time. As such, the compositions and methods described herein are uniquely suited to screening for HPV-associated malignancies, and to monitoring progression, treatment response, and post-treatment recurrence of HPV-associated tumors.

These results were achieved by rationally designing a series of novel optimized primers and probes—using a combination of three criteria. First, primers and probes were specifically designed to bind to regions of the HPV genome (within the E6 and/or E7 oncogenes) that are the most highly amplified in tumor genomes.18 Second, primers were specifically designed to generate amplicons that are significantly smaller than those that are typically used in other PCR-based HPV detection systems. The inventors hypothesized that, in view of the highly fragmented nature of ctDNA, small amplicon sizes would generate more reliable and consistent detection. Therefore, primers and primer pairs were designed specifically to generate amplicons that are significantly shorter than the average fragment length found in ctDNA and that are significantly shorter than those generated using current commercially available PCR-based HPV detection systems. Third, primers/probes were designed such that they exhibited a perfect match to both European and non-European HPV isolates—thereby maximizing universality of the test. Using the combination of these 3 design strategies, detection of HPV ctDNA with an unprecedented level of specificity and sensitivity was achieved—enabling even a single molecule of template HPV16 ctDNA to be detected in a droplet digital PCR assay (as shown in FIG. 1B), and outperforming the widely used commercially available Cobas® HPV test sold by Roche Molecular Diagnostics (as also shown in FIG. 1).

Building on these developments, the present invention provides a variety of new and improved compositions and methods for the diagnosis and monitoring of HPV-associated cancers—including a variety of rationally designed primers and probes, methods of detecting HPV DNA using such primers and probes, and methods for diagnosis and monitoring of HPV-associated cancers using such primers and probes.

For example, in one embodiment the present invention provides a method of detecting HPV circulating tumor DNA in a plasma sample from a subject, the method comprising: (a) performing a polymerase chain reaction comprising contacting a plasma sample obtained from a subject, or DNA obtained therefrom, with a primer pair consisting of a forward primer and a reverse primer, wherein the forward and reverse primer each bind to a target sequence in the HPV genome that is within the E6 and/or E7 region of the HPV genome, and is 100% conserved between European and non-European HPV isolates, and together span a region in the HPV genome that is about 175 nucleotides in length, or less, and (b) determining the presence of a specific amplified PCR product generated in the polymerase chain reaction, wherein if a specific amplified PCR product is detected, the plasma sample contains HPV circulating tumor DNA. Such a methods can be useful in a variety of situations. For example, in some embodiments such method steps provide a method of screening for an HPV-associated malignancy, wherein the presence of a specific amplified PCR product indicates that the subject has an HPV-associate malignancy. Similarly, in some embodiments such method steps provide assessing tumor burden of an HPV-positive tumor in a subject, wherein the quantity of the specific amplified PCR product correlates to tumor burden of the HPV-positive tumor in the subject.

In some embodiments the present invention provides variations of the above methods that are useful for monitoring changes in the level of HPV circulating tumor DNA over time. Such methods are particularly useful for monitoring the progression, recurrence, or response to therapy of an HPV-positive tumor in a subject. For example, in some embodiments the present invention provides a method for monitoring the progression, recurrence, or response to therapy of an HPV-positive tumor in a subject, the method comprising: (a) performing a polymerase chain reaction to detect HPV circulating tumor DNA in both a first plasma sample obtained from a subject at a first time point and a second plasma sample obtained from a subject at a second time point, or DNA obtained from such a plasma sample, using a primer pair consisting of a forward primer and a reverse primer, wherein the forward and reverse primer: each bind to a target sequence that is within the E6 and/or E7 region of the HPV genome, and is 100% conserved between European and non-European HPV isolates of HPV, and together span a region in the HPV genome that is about 100 nucleotides in length, or less, and (b) determining the quantity of a specific amplified PCR product generated in the polymerase chain reaction in both the first plasma sample and the second plasma sample, wherein an increase in the quantity of the specific amplified PCR product in the second sample as compared to the first sample indicates an increase in HPV-positive tumor burden from the first time point to the second time point, and a decrease in the quantity of the specific amplified PCR product in the second sample as compared to the first sample indicates a decrease in HPV-positive tumor burden from the first time point to the second time point.

In some embodiments of the methods described above and/or elsewhere herein, the HPV circulating tumor DNA is from a squamous cell carcinoma of the head and neck, oropharynx, cervix, vulva, vagina, anal canal or penis.

In some embodiments of the methods described above and/or elsewhere herein, the subject has, or is suspected of having, an HPV-associated malignancy/tumor that is a squamous cell carcinoma of the head and neck, oropharynx, cervix, vulva, vagina, anal canal or penis.

In some embodiments of the methods described above and/or elsewhere herein, the HPV circulating tumor DNA is DNA from HPV type 16, 18, 31, 33, 35, 39, 45, 51, 52, 56, 58 or 59.

In some embodiments of the methods described above and/or elsewhere herein, the HPV circulating tumor DNA is HPV16 DNA.

In some embodiments of the methods described above and/or elsewhere herein, the HPV circulating tumor DNA is HPV16 DNA and the subject has, or is suspected of having, a squamous cell carcinoma of the oropharynx or of the anal canal.

In some embodiments of the methods described above and/or elsewhere herein, the HPV DNA is HPV16 DNA, and the primer pair is selected from the group consisting of: (a) a forward primer comprising SEQ ID NO. 1 and a reverse primer comprising SEQ ID NO. 2, (b) a forward primer comprising SEQ ID NO. 9 and a reverse primer comprising SEQ ID NO. 10, (c) a forward primer comprising SEQ ID NO. 11 and a reverse primer comprising SEQ ID NO. 12, (d) a forward primer comprising SEQ ID NO. 13 and a reverse primer comprising SEQ ID NO. 14, (e) a forward primer comprising SEQ ID NO. 15 and a reverse primer comprising SEQ ID NO. 16, (f) a forward primer comprising SEQ ID NO. 17 and a reverse primer comprising SEQ ID NO. 18, (g) a forward primer comprising SEQ ID NO. 19 and a reverse primer comprising SEQ ID NO. 20, (h) a forward primer comprising SEQ ID NO. 21 and a reverse primer comprising SEQ ID NO. 22, (i) a forward primer comprising SEQ ID NO. 23 and a reverse primer comprising SEQ ID NO. 24, and (j) a forward primer comprising SEQ ID NO. 25 and a reverse primer comprising SEQ ID NO. 26.

In some embodiments of the methods described above and/or elsewhere herein, the HPV DNA is HPV33 DNA.

In some embodiments of the methods described above and/or elsewhere herein, the HPV DNA is HPV33 DNA and the subject has a squamous cell carcinoma of the oropharynx or of the anal canal.

In some embodiments of the methods described above and/or elsewhere herein, the HPV is HPV33, and the primer pair is selected from the group consisting of: (a) a forward primer comprising SEQ ID NO. 4 and a reverse primer comprising SEQ ID NO. 5, (b) a forward primer comprising SEQ ID NO. 36 and a reverse primer comprising SEQ ID NO. 37, (c) a forward primer comprising SEQ ID NO. 38 and a reverse primer comprising SEQ ID NO. 39, (d) a forward primer comprising SEQ ID NO. 40 and a reverse primer comprising SEQ ID NO. 41, (e) a forward primer comprising SEQ ID NO. 42 and a reverse primer comprising SEQ ID NO. 43, (f) a forward primer comprising SEQ ID NO. 44 and a reverse primer comprising SEQ ID NO. 45, (g) a forward primer comprising SEQ ID NO. 46 and a reverse primer comprising SEQ ID NO. 47, (h) a forward primer comprising SEQ ID NO. 48 and a reverse primer comprising SEQ ID NO. 49, (i) a forward primer comprising SEQ ID NO. 50 and a reverse primer comprising SEQ ID NO. 51, and (j) a forward primer comprising SEQ ID NO. 52 and a reverse primer comprising SEQ ID NO. 53.

In some embodiments of the methods described above and/or elsewhere herein, the HPV DNA is HPV18 DNA.

In some embodiments of the methods described above and/or elsewhere herein, the HPV DNA is HPV18 DNA and the subject has a squamous cell carcinoma of the head and neck, oropharynx, cervix, vulva, vagina, anal canal or penis.

In some embodiments of the methods described above and/or elsewhere herein, the HPV is HPV18, and the primer pair is selected from the group consisting of: (a) a forward primer comprising SEQ ID NO. 63 and a reverse primer comprising SEQ ID NO. 64, (b) a forward primer comprising SEQ ID NO. 65 and a reverse primer comprising SEQ ID NO. 66, (c) a forward primer comprising SEQ ID NO. 67 and a reverse primer comprising SEQ ID NO. 68, (d) a forward primer comprising SEQ ID NO. 69 and a reverse primer comprising SEQ ID NO. 70, (e) a forward primer comprising SEQ ID NO. 71 and a reverse primer comprising SEQ ID NO. 72, (f) a forward primer comprising SEQ ID NO. 73 and a reverse primer comprising SEQ ID NO. 74, (g) a forward primer comprising SEQ ID NO. 75 and a reverse primer comprising SEQ ID NO. 76, (h) a forward primer comprising SEQ ID NO. 77 and a reverse primer comprising SEQ ID NO. 78, (i) a forward primer comprising SEQ ID NO. 79 and a reverse primer comprising SEQ ID NO. 80, and (j) a forward primer comprising SEQ ID NO. 81 and a reverse primer comprising SEQ ID NO. 82.

The present invention also provides variations of the methods described above and/or elsewhere herein, in which more than one primer pair is used. For example, in some embodiments the methods of the present invention comprise performing the polymerase chain reaction using both a first primer pair and a second primer pair, or performing a first polymerase chain reaction using a first primer pair and a second polymerase chain reaction using a second primer pair, wherein both the first and second primer pairs consist of a forward primer and a reverse primer, wherein the forward and reverse primer each bind to a target sequence in the HPV genome that: is within the E6 and/or E7 region of the HPV genome, and is 100% conserved between European and non-European HPV isolates, and together span a region in the HPV genome that is about 175 nucleotides in length, or less, and wherein either: (a) the first primer pair is specific for HPV16 and the second primer pair is specific for HPV33, (b) the first primer pair is specific for HPV16 and the second primer pair is specific for HPV18, or (c) the first primer pair is specific for HPV18 and the second primer pair is specific for HPV33. Similarly, in a further variation of this method, the method comprises performing the polymerase chain reaction using a first primer pair, a second primer pair, and a third primer pair, or performing a first polymerase chain reaction using a first primer pair, a second polymerase chain reaction using a second primer pair and a third polymerase chain reaction using a third primer pair wherein each of the primer pairs consist of a forward primer and a reverse primer, wherein the forward and reverse primer: each bind to a target sequence in the HPV genome that: is within the E6 and/or E7 region of the HPV genome, and is 100% conserved between European and non-European HPV isolates, and together span a region in the HPV genome that is about 175 nucleotides in length, or less, and wherein the first primer pair is specific for HPV16, the second primer pair is specific for HPV33, and the third primer pair is specific for HPV18. In some of such embodiments the HPV16 primer pair is selected from the group consisting of: (a) a forward primer comprising SEQ ID NO. 1 and a reverse primer comprising SEQ ID NO. 2, (b) a forward primer comprising SEQ ID NO. 9 and a reverse primer comprising SEQ ID NO. 10, (c) a forward primer comprising SEQ ID NO. 11 and a reverse primer comprising SEQ ID NO. 12, (d) a forward primer comprising SEQ ID NO. 13 and a reverse primer comprising SEQ ID NO. 14, (e) a forward primer comprising SEQ ID NO. 15 and a reverse primer comprising SEQ ID NO. 16, (f) a forward primer comprising SEQ ID NO. 17 and a reverse primer comprising SEQ ID NO. 18, (g) a forward primer comprising SEQ ID NO. 19 and a reverse primer comprising SEQ ID NO. 20, (h) a forward primer comprising SEQ ID NO. 21 and a reverse primer comprising SEQ ID NO. 22, (i) a forward primer comprising SEQ ID NO. 23 and a reverse primer comprising SEQ ID NO. 24, and (j) a forward primer comprising SEQ ID NO. 25 and a reverse primer comprising SEQ ID NO. 26. In some of such embodiments the HPV33 primer pair is selected from the group consisting of: (a) a forward primer comprising SEQ ID NO. 4 and a reverse primer comprising SEQ ID NO. 5, (b) a forward primer comprising SEQ ID NO. 36 and a reverse primer comprising SEQ ID NO. 37, (c) a forward primer comprising SEQ ID NO. 38 and a reverse primer comprising SEQ ID NO. 39, (d) a forward primer comprising SEQ ID NO. 40 and a reverse primer comprising SEQ ID NO. 41, (e) a forward primer comprising SEQ ID NO. 42 and a reverse primer comprising SEQ ID NO. 43, (f) a forward primer comprising SEQ ID NO. 44 and a reverse primer comprising SEQ ID NO. 45, (g) a forward primer comprising SEQ ID NO. 46 and a reverse primer comprising SEQ ID NO. 47, (h) a forward primer comprising SEQ ID NO. 48 and a reverse primer comprising SEQ ID NO. 49, (i) a forward primer comprising SEQ ID NO. 50 and a reverse primer comprising SEQ ID NO. 51, and (j) a forward primer comprising SEQ ID NO. 52 and a reverse primer comprising SEQ ID NO. 53. In some of such embodiments the HPV18 primer pair is selected from the group consisting of: (a) a forward primer comprising SEQ ID NO. 63 and a reverse primer comprising SEQ ID NO. 64, (b) a forward primer comprising SEQ ID NO. 65 and a reverse primer comprising SEQ ID NO. 66, (c) a forward primer comprising SEQ ID NO. 67 and a reverse primer comprising SEQ ID NO. 68, (d) a forward primer comprising SEQ ID NO. 69 and a reverse primer comprising SEQ ID NO. 70, (e) a forward primer comprising SEQ ID NO. 71 and a reverse primer comprising SEQ ID NO. 72, (f) a forward primer comprising SEQ ID NO. 73 and a reverse primer comprising SEQ ID NO. 74, (g) a forward primer comprising SEQ ID NO. 75 and a reverse primer comprising SEQ ID NO. 76, (h) a forward primer comprising SEQ ID NO. 77 and a reverse primer comprising SEQ ID NO. 78, (i) a forward primer comprising SEQ ID NO. 79 and a reverse primer comprising SEQ ID NO. 80, and (j) a forward primer comprising SEQ ID NO. 81 and a reverse primer comprising SEQ ID NO. 82.

The embodiments of the invention described above and/or elsewhere herein involve using forward and reverse primers that bind to a target sequence in the HPV genome and together span a region in the HPV genome that is about 175 nucleotides in length, or less. In some embodiments of the methods described above and/or elsewhere herein the forward primer and the reverse primer span a region in the E6 and/or E7 region of the HPV genome that is about 150 nucleotides in length, or less. In some embodiments of the methods described above and/or elsewhere herein the forward primer and the reverse primer span a region in the E6 and/or E7 region of the HPV genome that is about 125 nucleotides in length, or less. In some embodiments of the methods described above and/or elsewhere herein the forward primer and the reverse primer span a region in the E6 and/or E7 region of the HPV genome that is about 100 nucleotides in length, or less. In some embodiments of the methods described above and/or elsewhere herein the forward primer and the reverse primer span a region in the E6 and/or E7 region of the HPV genome that is about 75 nucleotides in length, or less. In some embodiments of the methods described above and/or elsewhere herein the forward primer and the reverse primer span a region in the E6 and/or E7 region of the HPV genome that is about 97 nucleotides in length. Similarly, in some embodiments of the methods described above and/or elsewhere herein the specific amplified PCR product is about 150 nucleotides in length, or less. In some embodiments of the methods described above and/or elsewhere herein the specific amplified PCR product is about 125 nucleotides in length, or less. In some embodiments of the methods described above and/or elsewhere herein the specific amplified PCR product is about 100 nucleotides in length, or less. In some embodiments of the methods described above and/or elsewhere herein the specific amplified PCR product is about 75 nucleotides in length, or less. In some embodiments of the methods described above and/or elsewhere herein the specific amplified PCR product is about 97 nucleotides in length.

In some embodiments of the methods described above and/or elsewhere herein can be performed using any suitable PCR methodology or PCR technique known in the art. In some embodiments the PCR reaction is selected from a quantitative PCR reaction, a real-time quantitative PCR reaction, a digital PCR reaction, or a droplet digital PCR (ddPCR) reaction.

In some embodiments of the methods described above and/or elsewhere herein the PCR reaction is a ddPCR reaction.

In some embodiments of the methods described above and/or elsewhere herein, the amplified PCR products may be detected using any suitable method known in the art. In some embodiments the PCR products are detected by contacting them with a probe. In some such embodiments the probe may comprise a detectable moiety. In some such embodiments the probe may comprise a fluorophore. In some such embodiments the probe may comprise a fluorophore having a fluorescence property that changes upon hybridization. In some embodiments the amplified PCR products may be detected by contacting them with a probe that comprises a fluorophore and a quencher. In some of the embodiments described above and/or elsewhere herein, amplified PCR products from HPV16 DNA may be detected by contacting them with a probe that comprises SEQ ID. NO. 3, 27, 28, 29, 30, 31, 32, 33, 34, or 35. In some of the embodiments described above and/or elsewhere herein, amplified PCR products from HPV33 DNA may be detected by contacting them with a a probe that comprises SEQ ID. NO. 6, 54, 55, 56, 57, 58, 59, 60, 61, or 62. In some of the embodiments described above and/or elsewhere herein, amplified PCR products from HPV18 DNA may be detected by contacting them with a probe that comprises SEQ ID. NO. 83, 84, 85, 86, 87, 88, 89, 90, 91, or 92.

In some embodiments, any of the methods described above and/or elsewhere herein may also comprise a further step of treating the subject—for example with an anticancer therapeutic agent, with radiation therapy, with surgery, or any combination thereof.

In addition to the various methods described above and elsewhere herein, the present invention also comprises various compositions and kits.

For example, in some embodiments the present invention provides a composition or kit for detection of HPV16 DNA comprising one or more of the following primer pairs: (a) a forward primer comprising SEQ ID NO. 1 and a reverse primer comprising SEQ ID NO. 2, (b) a forward primer comprising SEQ ID NO. 9 and a reverse primer comprising SEQ ID NO. 10, (c) a forward primer comprising SEQ ID NO. 11 and a reverse primer comprising SEQ ID NO. 12, (d) a forward primer comprising SEQ ID NO. 13 and a reverse primer comprising SEQ ID NO. 14, (e) a forward primer comprising SEQ ID NO. 15 and a reverse primer comprising SEQ ID NO. 16, (f) a forward primer comprising SEQ ID NO. 17 and a reverse primer comprising SEQ ID NO. 18, (g) a forward primer comprising SEQ ID NO. 19 and a reverse primer comprising SEQ ID NO. 20, (h) a forward primer comprising SEQ ID NO. 21 and a reverse primer comprising SEQ ID NO. 22, (i) a forward primer comprising SEQ ID NO. 23 and a reverse primer comprising SEQ ID NO. 24, and (j) a forward primer comprising SEQ ID NO. 25 and a reverse primer comprising SEQ ID NO. 26. In some such embodiments such a composition or kit further comprises a probe that binds to an amplicon spanned by the primer pair. For example, in some embodiments the present invention provides a composition of kit for detection of HPV16 DNA comprising one or more of the following primer pair and probe combinations: (a) a forward primer comprising SEQ ID NO. 1 (Rama FOR primer) and a reverse primer comprising SEQ ID NO. 2 (Rama REV primer) and a probe comprising SEQ ID NO. 3, (b) a forward primer comprising SEQ ID NO. 9 (PR HPV16 primer1.FOR) and a reverse primer comprising SEQ ID NO. 10 (PR HPV16 primer1.REV) and a probe comprising SEQ ID NO.27, (c) a forward primer comprising SEQ ID NO. 11 and a reverse primer comprising SEQ ID NO. 12 and a probe comprising SEQ ID NO. 28, (d) a forward primer comprising SEQ ID NO. 13 and a reverse primer comprising SEQ ID NO. 14 and a probe comprising SEQ ID NO. 29, (e) a forward primer comprising SEQ ID NO. 15 and a reverse primer comprising SEQ ID NO. 16 and a probe comprising SEQ ID NO. 30, (f) a forward primer comprising SEQ ID NO. 17 and a reverse primer comprising SEQ ID NO. 18 and a probe comprising SEQ ID NO. 31, (g) a forward primer comprising SEQ ID NO. 19 and a reverse primer comprising SEQ ID NO. 20 and a probe comprising SEQ ID NO. 32, (h) a forward primer comprising SEQ ID NO. 21 and a reverse primer comprising SEQ ID NO. 22 and a probe comprising SEQ ID NO. 33, (i) a forward primer comprising SEQ ID NO. 23 and a reverse primer comprising SEQ ID NO. 24 and a probe comprising SEQ ID NO. 34, and (j) a forward primer comprising SEQ ID NO. 25 and a reverse primer comprising SEQ ID NO. 26 and a probe comprising SEQ ID NO. 35. In some such embodiments the probe comprises a detectable moiety. In some such embodiments the probe comprises a fluorophore. In some such embodiments the probe comprises a fluorophore having a fluorescence property that changes upon hybridization. In some of such embodiments the composition or kit further comprises an HPV16 positive control, an HPV16 negative control, or both HPV16 positive control and HPV16 negative control.

In some embodiments the present invention provides a composition or kit for detection of HPV33 DNA comprising one or more of the following primer pairs: (a) a forward primer comprising SEQ ID NO. 4 and a reverse primer comprising SEQ ID NO. 5, (b) a forward primer comprising SEQ ID NO. 36 and a reverse primer comprising SEQ ID NO. 37, (c) a forward primer comprising SEQ ID NO. 38 and a reverse primer comprising SEQ ID NO. 39, (d) a forward primer comprising SEQ ID NO. 40 and a reverse primer comprising SEQ ID NO. 41, (e) a forward primer comprising SEQ ID NO. 42 and a reverse primer comprising SEQ ID NO. 43, (f) a forward primer comprising SEQ ID NO. 44 and a reverse primer comprising SEQ ID NO. 45, (g) a forward primer comprising SEQ ID NO. 46 and a reverse primer comprising SEQ ID NO. 47, (h) a forward primer comprising SEQ ID NO. 48 and a reverse primer comprising SEQ ID NO. 49, (i) a forward primer comprising SEQ ID NO. 50 and a reverse primer comprising SEQ ID NO. 51, and (j) a forward primer comprising SEQ ID NO. 52 and a reverse primer comprising SEQ ID NO. 53. In some such embodiments such a composition or kit further comprises a probe that binds to an amplicon spanned by the primer pair. For example, in some embodiments the present invention provides a composition of kit for detection of HPV33 DNA comprising one or more of the following primer pair and probe combinations: (a) a forward primer comprising SEQ ID NO. 4 and a reverse primer comprising SEQ ID NO. 5 and a probe comprising SEQ ID NO. 6, (b) a forward primer comprising SEQ ID NO. 36 and a reverse primer comprising SEQ ID NO. 37 and a probe comprising SEQ ID NO. 54, (c) a forward primer comprising SEQ ID NO. 38 and a reverse primer comprising SEQ ID NO. 39 and a probe comprising SEQ ID NO. 55, (d) a forward primer comprising SEQ ID NO. 40 and a reverse primer comprising SEQ ID NO. 41 and a probe comprising SEQ ID NO. 56, (e) a forward primer comprising SEQ ID NO. 42 and a reverse primer comprising SEQ ID NO. 43 and a probe comprising SEQ ID NO. 57, (f) a forward primer comprising SEQ ID NO. 44 and a reverse primer comprising SEQ ID NO. 45 and a probe comprising SEQ ID NO. 58, (g) a forward primer comprising SEQ ID NO. 46 and a reverse primer comprising SEQ ID NO. 47 and a probe comprising SEQ ID NO. 59, (h) a forward primer comprising SEQ ID NO. 48 and a reverse primer comprising SEQ ID NO. 49 and a probe comprising SEQ ID NO. 60, (i) a forward primer comprising SEQ ID NO. 50 and a reverse primer comprising SEQ ID NO. 51 and a probe comprising SEQ ID NO. 61, (j) a forward primer comprising SEQ ID NO. 52 and a reverse primer comprising SEQ ID NO. 53 and a probe comprising SEQ ID NO. 62. In some such embodiments the probe comprises a detectable moiety. In some such embodiments the probe comprises a fluorophore. In some such embodiments the probe comprises a fluorophore having a fluorescence property that changes upon hybridization. In some of such embodiments the composition or kit further comprises an HPV33 positive control, an HPV33 negative control, or both an HPV33 positive control and an HPV33 negative control.

In some embodiments the present invention provides a composition or kit for detection of HPV18 DNA comprising one or more of the following primer pairs (a) a forward primer comprising SEQ ID NO. 63 and a reverse primer comprising SEQ ID NO. 64, (b) a forward primer comprising SEQ ID NO. 65 and a reverse primer comprising SEQ ID NO. 66, (c) a forward primer comprising SEQ ID NO. 67 and a reverse primer comprising SEQ ID NO. 68, (d) a forward primer comprising SEQ ID NO. 69 and a reverse primer comprising SEQ ID NO. 70, (e) a forward primer comprising SEQ ID NO. 71 and a reverse primer comprising SEQ ID NO. 72, (f) a forward primer comprising SEQ ID NO. 73 and a reverse primer comprising SEQ ID NO. 74, (g) a forward primer comprising SEQ ID NO. 75 and a reverse primer comprising SEQ ID NO. 76, (h) a forward primer comprising SEQ ID NO. 77 and a reverse primer comprising SEQ ID NO. 78, (i) a forward primer comprising SEQ ID NO. 79 and a reverse primer comprising SEQ ID NO. 80, and (j) a forward primer comprising SEQ ID NO. 81 and a reverse primer comprising SEQ ID NO. 82. In some such embodiments such a composition or kit further comprises a probe that binds to an amplicon spanned by the primer pair. For example, in some embodiments the present invention provides a composition of kit for detection of HPV18 DNA comprising one or more of the following primer pair and probe combinations: (a) a forward primer comprising SEQ ID NO. 63 and a reverse primer comprising SEQ ID NO. 64 and a probe comprising SEQ ID NO. 83, (b) a forward primer comprising SEQ ID NO. 65 and a reverse primer comprising SEQ ID NO. 66 and a probe comprising SEQ ID NO. 84, (c) a forward primer comprising SEQ ID NO. 67 and a reverse primer comprising SEQ ID NO. 68 and a probe comprising SEQ ID NO. 85, (d) a forward primer comprising SEQ ID NO. 69 and a reverse primer comprising SEQ ID NO. 70 and a probe comprising SEQ ID NO. 86, (e) a forward primer comprising SEQ ID NO. 71 and a reverse primer comprising SEQ ID NO. 72 and a probe comprising SEQ ID NO. 87, (f) a forward primer comprising SEQ ID NO. 73 and a reverse primer comprising SEQ ID NO. 74 and a probe comprising SEQ ID NO. 88, (g) a forward primer comprising SEQ ID NO. 75 and a reverse primer comprising SEQ ID NO. 76 and a probe comprising SEQ ID NO. 89, (h) a forward primer comprising SEQ ID NO. 77 and a reverse primer comprising SEQ ID NO. 78 and a probe comprising SEQ ID NO. 90, (i) a forward primer comprising SEQ ID NO. 79 and a reverse primer comprising SEQ ID NO. 80 and a probe comprising SEQ ID NO. 91, and (j) a forward primer comprising SEQ ID NO. 81 and a reverse primer comprising SEQ ID NO. 82 and a probe comprising SEQ ID NO. 92. In some such embodiments the probe comprises a detectable moiety. In some such embodiments the probe comprises a fluorophore. In some such embodiments the probe comprises a fluorophore having a fluorescence property that changes upon hybridization. In some of such embodiments the composition or kit further comprises an HPV18 positive control, an HPV18 negative control, or both an HPV18 positive control and an HPV18 negative control.

While some of the main embodiments of the present invention are summarized above, additional embodiments and additional details are provided and described in the Brief Description of the Figures, Detailed Description of the Invention, Examples, Claims, and Figures sections of this patent application. Furthermore, it should be understood that variations and combinations of each of the embodiments described above and elsewhere herein are contemplated and are intended to fall within the scope of the present invention.

### BRIEF DESCRIPTION OF THE DRAWINGS

FIG. 1A-E. Optimization of HPV16 and HPV33 ddPCR assays. (FIG. 1A) Standard curve for the Cobas® HPV Test using a dilution series of plasmid DNA containing the HPV16 genome (p1203 PML2d HPV-16). Each sample was tested in duplicate. (FIG. 1B) Standard curve for ddPCR using a dilution series of p1203 PML2d HPV-16, demonstrating single molecule sensitivity. Each sample was measured in triplicate. (FIG. 1C) Standard curve for ddPCR using serially diluted plasmid DNA containing HPV33 reference template. (FIG. 1D) Comparative analysis of ddPCR and Cobas® HPV Test for detection of HPV16 ctDNA in 8 patients with HNSCC. (FIG. 1E) Comparative analysis of ddPCR and qPCR for detecting HPV16 ctDNA in 12 HNSCC patients.

FIG. 2A-C. Sensitivity and specificity of HPV16/33 ddPCR assays. (FIG. 2A) HPV16 ctDNA detected by the HPV16 ddPCR assay in samples obtained from normal subjects (N=20) and patients with HPV negative head and neck cancers (N=7), HPV-associated oropharyngeal squamous cell carcinomas (N=97) and anal squamous cell carcinomas (N=8). Different shaped data points represent HPV16 pathological status as positive, negative and undetermined, respectively, as shown in the key. (FIG. 2B) HPV33 ddPCR assay was used for detecting HPV33 DNA in 7 patient cfDNA samples that did not have detectable HPV16 ctDNA. (FIG. 2C) Combination of both HPV16 and 33 ddPCR assays to determine overall sensitivity of HPV detection in pre-treatment samples from patients with HPV-associated OPSCC.

FIG. 3A-G. Factors influencing HPV16/33 ctDNA levels. (FIG. 3A) Relationship between HPV16/33 ctDNA in pretreatment samples and tumor volume. (FIG. 3B) Analysis of HPV16/33 ctDNA levels in small tumors. (FIG. 3C) Sequential measurements of HPV16 and KRAS G12D by ddPCR in a patient with a T1N2b HPV+OPSCC with a known KRAS G12D mutation. (FIG. 3D) Sequential measurements of HPV16 and PIK3CA E545K by ddPCR assays in a patient with T1N1 anal squamous cell carcinoma with a known PIK3CA E545K mutation. (FIG. 3E) Ratio of positive droplets for HPV16 and EIF2C1 ddPCR assays in genomic DNA samples extracted from tumor specimens as an estimate of HPV16 copy number per tumor cell genome. (FIG. 3F) Correlation between pretreatment levels of HPV measured by HPV16 ddPCR and HPV copy number per tumor genome. (FIG. 3G) Correlation between pretreatment levels of HPV measured by HPV16 ddPCR and product of HPV copy number and gross tumor volume.

FIG. 4A-D. HPV ddPCR assay in monitoring treatment response to chemoradiation. (FIG. 4A) Amplitude plots of the droplet digital PCR raw data are shown for weekly chemoradiation treatment (CRT) of one OPSCC patient, starting from pre-treatment sample on the left, weekly samples collected through 8 weeks of chemoradiation therapy and post treatment sample to the right. (FIG. 4B) Scatter plot depicting HPV16 copies/mL for each patient with pretreatment through post treatment samples (N=68 patients). (FIG. 4C) Graph representing median HPV copies/mL at each treatment time point from B along with interquartile range (black dotted lines), minimum and maximum values (gray dotted lines) in patients with complete sets of weekly plasma samples (N=28 patients). (FIG. 4D) Median HPV copies/mL measures in patient samples after every week of CRT are represented as a fraction of the same-patient pretreatment sample (N=28 patients). Gray dotted lines indicate minimum and maximum values, and black dotted lines show the interquartile range.

FIG. 5A-E. HPV ddPCR assay for measuring minimal residual disease. (FIG. 5A) Treatment course of a patient with T2N2b HPV+SCC of the right tonsil who complete definitive therapies 18 months prior to presentation with a paratracheal node recurrence (i). The patient received induction chemotherapy with resolution of disease by PET/CT (ii). The patient then received consolidative salvage SBRT to the involved area (iii). In 182 days following RT, a proximal subglottic nodule was discovered by PET/CT (iv). (FIG. 5B) P22 presented with a T3N2c HPV+OPSCC, which recurred four years later in the right tonsil, base of tongue, and right neck. The patient underwent a salvage resection, but before initiating adjuvant re-irradiation, developed recurrence in the right neck and pulmonary metastases. Despite palliative re-irradiation, HPV16 ctDNA levels increased and the pulmonary disease progressed while the right neck recurrence stabilized. (FIG. 5C) P56 presented with T4bN3M1 HPV+OPSCC of the right tonsil with metastatic disease in the right upper lobe, mediastinum and hilar nodes. Palliative RT was delivered to the bulk of visible disease in the head and neck, but HPV16 ctDNA levels changed only marginally while there was rapid progression in the chest and interval development of new osseous metastases. (FIG. 5D) Serial HPV16 ctDNA levels in three patients who recurred following chemoradiation. HPV16 ctDNA levels cleared to 0 droplets over the course of radiation yet developed distant metastases(DM) or local recurrences. (FIG. 5E) 9 patients with post-operative indications for adjuvant radiotherapy, including positive margins, extracapsular extension (ECE), or multiple positive nodes. In only one post-surgical sample (case 9) was HPV16 ctDNA detectable. In case 9, a neck dissection and non-definitive excisional biopsy was performed with all gross disease removed.

FIG. 6. Validation of HPV16 quantitative PCR assay. qPCR standard curve with serially diluted plasmid containing the HPV16 genome. This was the same plasmid used for generative standard curve for ddPCR assay in FIG. 1.

FIG. 7. HPV16 variant sequences.

### DETAILED DESCRIPTION OF THE INVENTION

The sub-headings provided below, and throughout this patent disclosure, are not intended to denote limitations of the various aspects or embodiments of the invention, which are to be understood by reference to the specification as-a-whole. For example, this Detailed Description is intended to read in conjunction with, and to expand upon, the description provided in all other sections of this patent application, including the Summary of the Invention and Examples sections thereof.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range. Where a numeric term is preceded by “about” or “approximately,” the term includes the stated number and values±20% of the stated number.

In all embodiments herein that refer to numeric value with the descriptor “about” (e.g. about 97 base pairs) alternate embodiments directed to the precise numeric value without the “about” descriptor are also contemplated and encompassed by the present invention.

Numbers in parentheses or superscript following text in this patent disclosure (particularly in the Background and Examples sections) refer to the numbered references provided in the “Reference List” section at the end of this patent disclosure.

Wherever embodiments are described with the language “comprising,” otherwise analogous embodiments described in terms of “consisting of” and/or “consisting essentially of” are also contemplated and encompassed by the present invention.

The term “increase” as used herein refers to any detectable increase. In some embodiments the increase is any statistically significant increase. In some embodiments the increase is an increase of about 10%, 20%, 30%, 40%, 50%, 60%, 70%, 80%, 90%, 100% or more.

The term “decrease” as used herein refers to any detectable decrease. In some embodiments the decrease is any statistically significant decrease. In some embodiments the decrease is a decrease of about 10%, 20%, 30%, 40%, 50%, 60%, 70%, 80%, 90% or 100%.

In all embodiments that refer to one or more primer pairs, specific embodiments that involve 1, 2, 3, 4, 5, 6, 7, 8, 9, or 10 of such primer pairs are also contemplated and encompassed by the present invention.

In all embodiments that refer to one or more primer pair and probe combinations, specific embodiments that comprise 1, 2, 3, 4, 5, 6, 7, 8, 9, or 10 of such primer pair and probe combinations are also contemplated and encompassed by the present invention.

Other abbreviations and definitions may be provided elsewhere in this patent specification, or may be well known in the art.

Numbers in parentheses or superscript following text in this patent disclosure (particularly in the Background and Examples sections) refer to the numbered references provided in the “Reference List” section at the end of this patent disclosure

In some embodiments the present invention provides various methods of detecting HPV circulating tumor DNA in a plasma sample from a subject. In some embodiments such methods involve performing a polymerase chain reaction in which a plasma sample obtained from a subject (or DNA obtained from such a plasma sample) is contacted with a forward and reverse primer that each bind to a target sequence that is within the E6 and/or E7 region of the HPV genome and is 100% conserved between European and non-European HPV isolates, and that together span a region in the HPV genome that is about 175 nucleotides in length, or less, and then determining the presence, absence or quantity of a specific amplified PCR product generated in the polymerase chain reaction. In such methods, detection of the specific amplified PCR product indicates that the plasma sample contains HPV circulating tumor DNA. Many of such methods are described in the Summary of the Invention section of this patent specification and elsewhere in this patent specification. In such methods the forward and reverse primer will typically comprise a sequence that is exactly complementary to the region of the target sequence to which it is designed to bind. However, in some embodiments, there may be one or more mismatches provided that the primer retains the ability to bind to the target sequence under high stringency conditions.

The methods described herein have a variety of applications. For example, in some embodiments such methods can be employed to screen for HPV-associated malignancies (such as an HPV-positive squamous cell carcinomas) whereby the presence of the specific amplified PCR product indicates that the subject has an HPV-associated malignancy. Similarly, in some embodiments such methods can be employed to assess tumor burden of an HPV-positive cancer, such as an HPV-positive squamous cell carcinoma, in a subject—as the quantity of the specific amplified PCR product correlates to tumor burden. In some of such methods controls and/or standard curves are used to quantify or give an estimate of the tumor burden—e.g. in terms of tumor volume or number or tumor cells, etc. Similarly, in other embodiments such methods can be employed to monitor the progression or recurrence of an HPV-positive cancer, such as an HPV-positive squamous cell carcinoma, in a subject, or to monitor the response to therapy of an HPV-positive cancer, such as an HPV-positive squamous cell carcinoma, in a subject. Such methods involve determining changes in the quantity of the specific amplified PCR products over time. Typically, such methods entail performing the PCR methods using two or more plasma samples obtained from the subject at different time points. For example, in some embodiments a first plasma sample is obtained from the subject at a first time point and a second plasma sample is obtained from the subject at a second time point. Using such methods, an increase or decrease in the quantity of the specific amplified PCR product between the first sample/time point and the second sample/time point can be informative. For example, an increase in the quantity of the specific amplified PCR product between the first sample/time point the second sample/time point may indicate an increase tumor burden, for example as a result of tumor progression, or as a result of tumor recurrence following a previous treatment.

Similarly, a decrease in the quantity of the specific amplified PCR product between a first sample/time point prior to treatment (or earlier in treatment) and a second sample/time point subsequent to commencement of treatment (or later in treatment, or after treatment) may indicate that the treatment is effective. Conversely, an increase in the quantity of the specific amplified PCR product between a first sample/time point prior to treatment (or earlier in treatment) and a second sample/time point subsequent to commencement of treatment (or later in treatment, or after treatment) may indicate that the treatment is ineffective. In those methods aimed monitoring the response to therapy, the methods may be performed using both a “test” sample and a “control” sample. For example, the test sample may be obtained from a subject treated with a new/test therapeutic molecule and the control sample may be obtained from an untreated subject, or a subject treated with a placebo, or a subject treated with a comparator therapeutic molecule. Such methods can be used to monitor the response to any desired type of therapy, including, but not limited to, therapy with chemotherapeutic agents, therapy with other therapeutic molecules, therapy using radiation, and surgical therapy.

In each of the embodiments summarized above, and described elsewhere herein, the HPV circulating tumor DNA can be that from any HPV-associated cancer. In some embodiments the HPV-associated cancer is a squamous cell carcinoma. In some such embodiments the HPV-associated cancer is selected from the group consisting of squamous cell carcinoma of the head and neck, oropharynx, cervix, vulva, vagina, anal canal or penis. In some such embodiments the HPV-associated cancer is a squamous cell carcinoma of the oropharynx. In some such embodiments the HPV-associated cancer is a squamous cell carcinoma of the anal canal. Similarly, in each of the embodiments summarized above, and described elsewhere herein, the subject may have, or be suspected of having any HPV-associated cancer. In some such embodiments the subject may have, or be suspected of having, an HPV-associated squamous cell carcinoma, such as an HPV-associated squamous cell carcinoma selected from the group consisting of squamous cell carcinoma of the head and neck, oropharynx, cervix, vulva, vagina, anal canal or penis. In some such embodiments the HPV-associated cancer is a squamous cell carcinoma of the oropharynx. In some such embodiments the HPV-associated cancer is a squamous cell carcinoma of the anal canal.

In each of the embodiments summarized above, and described elsewhere herein, the HPV circulating tumor DNA can be from any HPV type. In some such embodiments, the HPV circulating tumor DNA is from HPV type 16, 18, 31, 33, 35, 39, 45, 51, 52, 56, 58 or 59. In some such embodiments, the HPV circulating tumor DNA is from HPV16. In some such embodiments, the HPV circulating tumor DNA is from HPV33. In some such embodiments, the HPV circulating tumor DNA is from HPV18.

In some of the embodiments summarized above, and described elsewhere herein, the HPV circulating tumor DNA is HPV16 DNA and the subject has, or is suspected of having, a squamous cell carcinoma of the oropharynx or of the anal canal.

In some of the embodiments summarized above, and described elsewhere herein, the HPV circulating tumor DNA is HPV33 DNA and the subject has, or is suspected of having, a squamous cell carcinoma of the oropharynx or of the anal canal.

In some of the embodiments summarized above, and described elsewhere herein, the HPV circulating tumor DNA is HPV 18 DNA and the subject has, or is suspected of having, a squamous cell carcinoma of the head and neck, oropharynx, cervix, vulva, vagina, anal canal or penis.

I. HPV16 Primer Pairs

In some of the embodiments summarized above, and described elsewhere herein, HPV16 DNA (e.g. ctDNA) may be detected using one or more of the following primer pairs (see Table 1 below for sequences): (1) a forward primer comprising SEQ ID NO. 1 (Rama FOR primer) and a reverse primer comprising SEQ ID NO. 2 (Rama REV primer), (2) a forward primer comprising SEQ ID NO. 9 (PR HPV16 primer1.FOR) and a reverse primer comprising SEQ ID NO. 10 (PR HPV16 primer1.REV), (3) a forward primer comprising SEQ ID NO. 11 and a reverse primer comprising SEQ ID NO. 12, (4) a forward primer comprising SEQ ID NO. 13 and a reverse primer comprising SEQ ID NO. 14, (5) a forward primer comprising SEQ ID NO. 15 and a reverse primer comprising SEQ ID NO. 16, (6) a forward primer comprising SEQ ID NO. 17 and a reverse primer comprising SEQ ID NO. 18, (7) a forward primer comprising SEQ ID NO. 19 and a reverse primer comprising SEQ ID NO. 20, (8) a forward primer comprising SEQ ID NO. 21 and a reverse primer comprising SEQ ID NO. 22, (9) a forward primer comprising SEQ ID NO. 23 and a reverse primer comprising SEQ ID NO. 24, and/or (10) a forward primer comprising SEQ ID NO. 25 and a reverse primer comprising SEQ ID NO. 26.

In some embodiments the present invention also provides compositions and kits comprising one or more of such HPV16 primer pairs.

II. HPV16 Primer & Probe Combinations

In some of the methods summarized above, and described elsewhere herein, HPV16 DNA, such as amplified HPV16 PCR products, are detected with a probe. In some such embodiments the HPV16 DNA may be detected by performing a PCR reaction and detection step using one or more of the following combinations of primers and probes (see Table 1 below for sequences): (1) a forward primer comprising SEQ ID NO. 1 (Rama FOR primer) and a reverse primer comprising SEQ ID NO. 2 (Rama REV primer) and a probe comprising SEQ ID NO. 3, (2) a forward primer comprising SEQ ID NO. 9 (PR HPV16 primer1.FOR) and a reverse primer comprising SEQ ID NO. 10 (PR HPV16 primer1.REV) and a probe comprising SEQ ID NO.27, (3) a forward primer comprising SEQ ID NO. 11 and a reverse primer comprising SEQ ID NO. 12 and a probe comprising SEQ ID NO. 28, (4) a forward primer comprising SEQ ID NO. 13 and a reverse primer comprising SEQ ID NO. 14 and a probe comprising SEQ ID NO. 29, (5) a forward primer comprising SEQ ID NO. 15 and a reverse primer comprising SEQ ID NO. 16 and a probe comprising SEQ ID NO. 30, (6) a forward primer comprising SEQ ID NO. 17 and a reverse primer comprising SEQ ID NO. 18 and a probe comprising SEQ ID NO. 31, (7) a forward primer comprising SEQ ID NO. 19 and a reverse primer comprising SEQ ID NO. 20 and a probe comprising SEQ ID NO. 32, (8) a forward primer comprising SEQ ID NO. 21 and a reverse primer comprising SEQ ID NO. 22 and a probe comprising SEQ ID NO. 33, (9) a forward primer comprising SEQ ID NO. 23 and a reverse primer comprising SEQ ID NO. 24 and a probe comprising SEQ ID NO. 34, and/or (10) a forward primer comprising SEQ ID NO. 25 and a reverse primer comprising SEQ ID NO. 26 and a probe comprising SEQ ID NO. 35.

In some embodiments the present invention also provides compositions and kits comprising one or more of such HPV16 primer pair and probe combinations.

TABLE 1
HPV 16 Primers & Probes

| Primer/Probe Name | Sequence | SEQ ID NO. |
|---|---|---|
| HPV16 forward primer | tatgcacagagctgcaaaca | SEQ ID NO. 1 |
| (Rama FOR primer) | | |
| HPV16 reverse primer | gcaaagtcatatacctcacgtc | SEQ ID NO. 2 |
| (Rama REV primer) | | |
| HPV16 probe | tgtgtgtactgcaagcaacagttactg | SEQ ID NO. 3 |
| (Rama Probe) | | |
| HPV16 forward (qPCR) | cggtcgatgtatgtcttgtt | SEQ ID NO. 7 |
| HPV16 reverse(qPCR) | ctgggtttctctacgtgttc | SEQ ID NO. 8 |
| PR HPV16 primer1.FOR | tgcaccaaaagagaactgc | SEQ ID NO. 9 |
| PR HPV16 primer1.REV | tgtttgcagctctgtgcata | SEQ ID NO. 10 |
| PR HPV16 primer2.FOR | ggaacaacattagaacagcaatac | SEQ ID NO. 11 |
| PR HPV16 primer2.REV | gtccagatgtctttgcttttc | SEQ ID NO. 12 |
| PR HPV16 primer3.FOR | agcaaagattccataatataaggg | SEQ ID NO. 13 |
| PR HPV16 primer3.REV | ttacagctgggtttctctacg | SEQ ID NO. 14 |
| PR HPV16 primer4.FOR | gcatggagatacacctacat | SEQ ID NO. 15 |
| PR HPV16 primer4.REV | ttaattgctcataacagtagagatcag | SEQ ID NO. 16 |
| PR HPV16 primer5.FOR | acaactgatctctactgttatgag | SEQ ID NO. 17 |
| PR HPV16 primer5.REV | ggttacaatattgtaatgggctc | SEQ ID NO. 18 |
| PR HPV16 primer6.FOR | agaaccggacagagcc | SEQ ID NO. 19 |
| PR HPV16 primer6.REV | gtctacgtgtgtgctttgtac | SEQ ID NO. 20 |
| PR HPV16 primer7.FOR | gactctacgcttcggtt | SEQ ID NO. 21 |
| PR HPV16 primer7.REV | gaacagatggggcacac | SEQ ID NO. 22 |
| PR HPV16 primer8.FOR | tagaatgtgtgtactgcaagc | SEQ ID NO. 23 |
| PR HPV16 primer8.REV | cactaattttagaataaaactttaaacatttatcac | SEQ ID NO. 24 |
| PR HPV16 primer9.FOR | gtgataaatgtttaaagttttattctaaaattagtg | SEQ ID NO. 25 |
| PR HPV16 primer9.REV | ggcttttgacagttaatacacc | SEQ ID NO. 26 |
| PR HPV16 probe1 | aggacccacaggagcgac | SEQ ID NO. 27 |
| PR HPV16 probe2 | attaggtgtattaactgtcaaaagccact | SEQ ID NO. 28 |
| PR HPV16 probe3 | tggaccggtcgatgtatgtcttgtt | SEQ ID NO. 29 |
| PR HPV16 probe4 | tgcatgaatatatgttagatttgcaaccagag | SEQ ID NO. 30 |
| PR HPV16 probe5 | aaatagatggtccagctggacaagc | SEQ ID NO. 31 |
| PR HPV16 probe6 | ttgcaagtgtgactctacgcttcg | SEQ ID NO. 32 |
| PR HPV16 probe7 | ttggaagacctgttaatgggcacac | SEQ ID NO. 33 |
| PR HPV16 probe8 | ttttcgggatttatgcatagtatatagagatggg | SEQ ID NO. 34 |
| PR HPV16 probe9 | tgtatggaacaacattagaacagcaatacaacaaac | SEQ ID NO. 35 |

III. HPV33 Primer Pairs

In some of the embodiments summarized above, and described elsewhere herein, HPV33 DNA (e.g. ctDNA) may be detected using one or more of the following primer pairs (see Table 2 below for sequences): (1) a forward primer comprising SEQ ID NO. 4 and a reverse primer comprising SEQ ID NO. 5, (2) a forward primer comprising SEQ ID NO. 36 and a reverse primer comprising SEQ TD NO. 37, (3) a forward primer comprising SEQ ID NO. 38 and a reverse primer comprising SEQ ID NO. 39, (4) a forward primer comprising SEQ ID NO. 40 and a reverse primer comprising SEQ ID NO. 41, (5) a forward primer comprising SEQ TD NO. 42 and a reverse primer comprising SEQ ID NO. 43, (6) a forward primer comprising SEQ ID NO. 44 and a reverse primer comprising SEQ TD NO. 45, (7) a forward primer comprising SEQ ID NO. 46 and a reverse primer comprising SEQ ID NO. 47, (8) a forward primer comprising SEQ TD NO. 48 and a reverse primer comprising SEQ ID NO. 49, (9) a forward primer comprising SEQ ID NO. 50 and a reverse primer comprising SEQ ID NO. 51, and/or (10) a forward primer comprising SEQ TD NO. 52 and a reverse primer comprising SEQ ID NO. 53.

In some embodiments the present invention also provides compositions and kits comprising one or more of such HPV33 primer pairs.

IV. HPV33 Primer & Probe Combinations

In some of the methods summarized above, and described elsewhere herein, HPV33 DNA (e.g. amplified ctDNA HPV33 PCR products) is detected with a probe. In some such embodiments the HPV33 DNA may be detected by performing a PCR reaction and detection using one or more of the following combinations of primers and probes (see Table 2 below for sequences): (1) a forward primer comprising SEQ ID NO. 4 and a reverse primer comprising SEQ ID NO. 5 and a probe comprising SEQ ID NO. 6, (2) a forward primer comprising SEQ ID NO. 36 and a reverse primer comprising SEQ ID NO. 37 and a probe comprising SEQ ID NO. 54, (3) a forward primer comprising SEQ ID NO. 38 and a reverse primer comprising SEQ ID NO. 39 and a probe comprising SEQ ID NO. 55, (4) a forward primer comprising SEQ ID NO. 40 and a reverse primer comprising SEQ ID NO. 41 and a probe comprising SEQ ID NO. 56, (5) a forward primer comprising SEQ ID NO. 42 and a reverse primer comprising SEQ ID NO. 43 and a probe comprising SEQ ID NO. 57, (6) a forward primer comprising SEQ ID NO. 44 and a reverse primer comprising SEQ ID NO. 45 and a probe comprising SEQ ID NO. 58, (7) a forward primer comprising SEQ ID NO. 46 and a reverse primer comprising SEQ ID NO. 47 and a probe comprising SEQ ID NO. 59, (8) a forward primer comprising SEQ ID NO. 48 and a reverse primer comprising SEQ ID NO. 49 and a probe comprising SEQ ID NO. 60, (9) a forward primer comprising SEQ ID NO. 50 and a reverse primer comprising SEQ ID NO. 51 and a probe comprising SEQ ID NO. 61, and/or (10) a forward primer comprising SEQ ID NO. 52 and a reverse primer comprising SEQ ID NO. 53 and a probe comprising SEQ ID NO. 62.

In some embodiments the present invention also provides compositions and kits comprising one or more of such HPV33 primer pair and probe combinations.

TABLE 2
HPV 33 Primers & Probes

| Primer/Probe Name | Sequence | SEQ ID NO. |
|---|---|---|
| HPV33 forward primer | ccacagttcgtttatgtgtca | SEQ ID NO. 4 |
| HPV33 reverse primer | tgcccataagtagttgctgt | SEQ ID NO. 5 |
| HPV33 probe | agtacagcaagtgacctacgaacca- | SEQ ID NO. 6 |
| PR HPV33 primer1.FOR | atgtttcaagacactgaggaaaa | SEQ ID NO. 36 |
| PR HPV33 primer1.REV | ccacgcactgtagttcaa | SEQ ID NO. 37 |
| PR HPV33 primer2.FOR | ggagacaactatacacaacattg | SEQ ID NO. 38 |
| PR HPV33 primer2.REV | ctatatacaactgttaaatctgcaaatg | SEQ ID NO. 39 |
| PR HPV33 primer3.FOR | gcatttgcagatttaacagttgtatatag | SEQ ID NO. 40 |
| PR HPV33 primer3.REV | ctatattcactaattttagataagaaccgc | SEQ ID NO. 41 |
| PR HPV33 primer4.FOR | aaacctttaaatgaaatattaattaggtgtat | SEQ ID NO. 42 |
| PR HPV33 primer4.REV | ccaacgacccgaaatattatg | SEQ ID NO. 43 |
| PR HPV33 primer5.FOR | cataatatttcgggtcgttgg | SEQ ID NO. 44 |
| PR HPV33 primer5.REV | gtgtcctctcatggcg | SEQ ID NO. 45 |
| PR HPV33 primer6.FOR | agccaacgttaaaggaatatg | SEQ ID NO. 46 |
| PR HPV33 primer6.REV | ctgagctgtcacttaattgc | SEQ ID NO. 47 |
| PR HPV33 primer7.FOR | tgagcaattaagtgacagctc | SEQ ID NO. 48 |
| PR HPV33 primer7.REV | gctgtggctggttgt | SEQ ID NO. 49 |
| PR HPV33 primer8.FOR | gatggacaagcacaacca | SEQ ID NO. 50 |
| PR HPV33 primer8.REV | tatggttcgtaggtcacttgc | SEQ ID NO. 51 |
| PR HPV33 primer9.FOR | gtaacaccacagttcgtttatg | SEQ ID NO. 52 |
| PR HPV33 primer9.REV | actgtgcccataagtagttg | SEQ ID NO. 53 |
| PR HPV33 probe1 | acattgcatgatttgtgccaagc | SEQ ID NO. 54 |
| PR HPV33 probe2 | cctttgcaacgatctgaggtatatgattt | SEQ ID NO. 55 |
| PR HPV33 probe3 | agggaaatccatttggaatatgtaaactgtg | SEQ ID NO. 56 |
| PR HPV33 probe4 | agaaaaaaaacgacatgtggatttaaacaaacg | SEQ ID NO. 57 |
| PR HPV33 probe5 | cgacgtagagaaactgcactgtg | SEQ ID NO. 58 |
| PR HPV33 probe6 | agatttatatcctgaaccaactgacctatactgc | SEQ ID NO. 59 |
| PR HPV33 probe7 | tgaggatgaaggcttggaccg | SEQ ID NO. 60 |
| PR HPV33 probe8 | ccacagttcgtttatgtgtcaacagt | SEQ ID NO. 61 |
| PR HPV33 probe9 | gcaagtgacctacgaaccatacag | SEQ ID NO. 62 |

V. HPV18 Primer Pairs

In some of the embodiments summarized above, and described elsewhere herein, HPV18 DNA (e.g. HPV18 ctDNA) may be detected using one or more of the following primer pairs (see Table 3 below for sequences): (1) a forward primer comprising SEQ ID NO. 63 and a reverse primer comprising SEQ ID NO. 64, (2) a forward primer comprising SEQ ID NO. 65 and a reverse primer comprising SEQ ID NO. 66, (3) a forward primer comprising SEQ ID NO. 67 and a reverse primer comprising SEQ ID NO. 68, (4) a forward primer comprising SEQ ID NO. 69 and a reverse primer comprising SEQ ID NO. 70, (5) a forward primer comprising SEQ ID NO. 71 and a reverse primer comprising SEQ ID NO. 72, (6) a forward primer comprising SEQ ID NO. 73 and a reverse primer comprising SEQ ID NO. 74, (7) a forward primer comprising SEQ ID NO. 75 and a reverse primer comprising SEQ ID NO. 76, (8) a forward primer comprising SEQ ID NO. 77 and a reverse primer comprising SEQ ID NO. 78, (9) a forward primer comprising SEQ ID NO. 79 and a reverse primer comprising SEQ ID NO. 80, and/or (10) a forward primer comprising SEQ ID NO. 81 and a reverse primer comprising SEQ ID NO. 82.

In some embodiments the present invention also provides compositions and kits comprising one or more of such HPV18 primer pairs.

VI. HPV18 Primer & Probe Combinations

In some of the methods summarized above, and described elsewhere herein, HPV18 DNA (e.g. an amplified HPV18 ctDNA PCR product) is detected with a probe. In some such embodiments the HPV18 DNA may be detected by performing a PCR reaction and detection using one or more of the following combinations of primers and probes (see Table 3 below for sequences): (1) a forward primer comprising SEQ ID NO. 63 and a reverse primer comprising SEQ ID NO. 64 and a probe comprising SEQ ID NO. 83, (2) a forward primer comprising SEQ ID NO. 65 and a reverse primer comprising SEQ ID NO. 66 and a probe comprising SEQ ID NO. 84, (3) a forward primer comprising SEQ ID NO. 67 and a reverse primer comprising SEQ ID NO. 68 and a probe comprising SEQ ID NO. 85, (4) a forward primer comprising SEQ ID NO. 69 and a reverse primer comprising SEQ ID NO. 70 and a probe comprising SEQ TD NO. 86, (5) a forward primer comprising SEQ ID NO. 71 and a reverse primer comprising SEQ TD NO. 72 and a probe comprising SEQ ID NO. 87, (6) a forward primer comprising SEQ TD NO. 73 and a reverse primer comprising SEQ ID NO. 74 and a probe comprising SEQ ID NO. 88, (7) a forward primer comprising SEQ ID NO. 75 and a reverse primer comprising SEQ ID NO. 76 and a probe comprising SEQ ID NO. 89, (8) a forward primer comprising SEQ ID NO. 77 and a reverse primer comprising SEQ ID NO. 78 and a probe comprising SEQ ID NO. 90, (9) a forward primer comprising SEQ ID NO. 79 and a reverse primer comprising SEQ ID NO. 80 and a probe comprising SEQ ID NO. 91, and/or (10) a forward primer comprising SEQ ID NO. 81 and a reverse primer comprising SEQ TD NO. 82 and a probe comprising SEQ ID NO. 92.

In some embodiments the present invention also provides compositions and kits comprising one or more of such HPV18 primer pair and probe combinations.

TABLE 3
HPV18 Primers & Probes

| Primer/Probe Name | Sequence | SEQ ID NO. |
|---|---|---|
| PR HPV18 primer1.FOR | atggcgcgctttga | SEQ ID NO. 63 |
| PR HPV18 primer1.REV | ctatgtcttgcagtgaagtgtt | SEQ ID NO. 64 |
| PR HPV18 primer2.FOR | tctgtgcacggaactga | SEQ ID NO. 65 |
| PR HPV18 primer2.REV | ctgtaagttccaatactgtcttg | SEQ ID NO. 66 |
| PR HPV18 primer3.FOR | gtgtatattgcaagacagtattgg | SEQ ID NO. 67 |
| PR HPV18 primer3.REV | ctctaattctagaataaaaatctatacatttatggc | SEQ ID NO. 68 |
| PR HPV18 primer4.FOR | attattcagactctgtgtatggag | SEQ ID NO. 69 |
| PR HPV18 primer4.REV | gtgtctaagtttttctgctgg | SEQ ID NO. 70 |
| PR HPV18 primer5.FOR | gcggtgccagaaacc | SEQ ID NO. 71 |
| PR HPV18 primer5.REV | ggttgcagcacgaatgg | SEQ ID NO. 72 |
| PR HPV18 primer6.FOR | gacattgtattgcatttagagcc | SEQ ID NO. 73 |
| PR HPV18 primer6.REV | aactccatctatttcatcgttttc | SEQ ID NO. 74 |
| PR HPV18 primer7.FOR | caattaagcgactcagaggaag | SEQ ID NO. 75 |
| PR HPV18 primer7.REV | cattgtgtgacgttgtgg | SEQ ID NO. 76 |
| PR HPV18 primer8.FOR | accacaacgtcacacaat | SEQ ID NO. 77 |
| PR HPV18 primer8.REV | tgctgagctttctactactagc | SEQ ID NO. 78 |
| PR HPV18 primer9.FOR | gctagtagtagaaagctcagc | SEQ ID NO. 79 |
| PR HPV18 primer9.REV | acaccacggacacac | SEQ ID NO. 80 |
| PR HPV18 primer10.FOR | ggcgaccctacaagc | SEQ ID NO. 81 |
| PR HPV18 primer10.REV | ctgtcttgcaatatacacagg | SEQ ID NO. 82 |
| PR HPV18 probe1 | tccaacacggcgaccctac | SEQ ID NO. 83 |
| PR HPV18 probe2 | cacttcactgcaagacatagaaataacctg | SEQ ID NO. 84 |
| PR HPV18 probe3 | tatagagacagtataccgcatgctgca | SEQ ID NO. 85 |
| PR HPV18 probe4 | ctgggttatacaatttattaataaggtgcctgc | SEQ ID NO. 86 |
| PR HPV18 probe5 | ctgggcactatagaggccagt | SEQ ID NO. 87 |
| PR HPV18 probe6 | cgagcaattaagcgactcagagg | SEQ ID NO. 88 |
| PR HPV18 probe7 | agaaaacgatgaaatagatggagttaatcatcaacatt | SEQ ID NO. 89 |
| PR HPV18 probe8 | tgtatgtgttgtaagtgtgaagccagaa | SEQ ID NO. 90 |
| PR HPV18 probe9 | cgaccttcgagcattccagc | SEQ ID NO. 91 |
| PR HPV18 probe10 | ctgtgcacggaactgaacactt | SEQ ID NO. 92 |

In some of the embodiments summarized above, and described elsewhere herein, the HPV DNA (e.g. ctDNA) is or comprises both HPV16 DNA and HPV33 DNA, or both HPV16 DNA and HPV18 DNA, or both HPV18 DNA and HPV33 DNA, or HPV16 DNA, HPV18 DNA and HPV33 DNA. In such cases each of these DNAs can be detected using a combination of the HPV16 DNA, HPV18 DNA and HPV33 DNA primer pairs and/or probes described above. Similarly, in some embodiments the present invention also provides compositions and kits comprising one or more of the HPV16, HPV18 and/or HPV33 primer pair pairs, or primer pair and probe combinations, described above.

One aspect of the methods of the present invention is that they may involve the amplification (e.g. by PCR) of regions of HPV DNA (amplicons) that are shorter than those amplified using some prior methods. As summarized above, and as described elsewhere herein, in some embodiments the forward primer and the reverse primer span a region in the E6 and/or E7 region of the HPV genome that is about 150 nucleotides in length, or less, and/or result in the generation of a specific amplified PCR product that is about 150 nucleotides in length, or less. In some embodiments the forward primer and the reverse primer span a region in the E6 and/or E7 region of the HPV genome that is about 125 nucleotides in length, or less, and/or result in the generation of a specific amplified PCR product that is about 125 nucleotides in length, or less. In some embodiments the forward primer and the reverse primer span a region in the E6 and/or E7 region of the HPV genome that is about 100 nucleotides in length, or less, and/or result in the generation of a specific amplified PCR product that is about 100 nucleotides in length, or less. In some embodiments the forward primer and the reverse primer span a region in the E6 and/or E7 region of the HPV genome that is about 75 nucleotides in length, or less, and/or result in the generation of a specific amplified PCR product that is about 75 nucleotides in length, or less. In some embodiments the forward primer and the reverse primer span a region in the E6 and/or E7 region of the HPV genome that is about 97 nucleotides in length, and/or result in the generation of a specific amplified PCR product that is about 97 nucleotides in length. In some embodiments the forward primer and the reverse primer span a region in the E6 and/or E7 region of the HPV genome that is at least about 10 nucleotides in length, or at least about 20 nucleotides in length, at least about 30 nucleotides in length, at least about 40 nucleotides in length, at least about 50 nucleotides in length, at least about 60 nucleotides in length, at least about 70 nucleotides in length, at least about 80 nucleotides in length, at least about 90 nucleotides in length. In some embodiments the forward primer and the reverse primer, when used in a PCR reaction, result in the generation of a specific amplified PCR product that is at least about 10 nucleotides in length, or at least about 20 nucleotides in length, at least about 30 nucleotides in length, at least about 40 nucleotides in length, at least about 50 nucleotides in length, at least about 60 nucleotides in length, at least about 70 nucleotides in length, at least about 80 nucleotides in length, at least about 90 nucleotides in length.

In some embodiments any of the primers and/or probes described herein may comprise one or more non-naturally occurring nucleotides. In some embodiments any of the primers and/or probes described herein may comprise one or more detectable moieties, such as one or more fluorescent moieties.

In each of the embodiments summarized above, and described elsewhere herein, that involve a PCR reaction, the PCR method can be any suitable PCR method. In some embodiments the PCR method is a quantitative PCR method, a real-time quantitative PCR method, a digital PCR method or a droplet digital PCR (ddPCR) method. In some embodiments the PCR method is a droplet digital PCR (ddPCR) method.

In some of the methods summarized above, and described elsewhere herein, the amplified PCR products are detected with a probe. In some such embodiments the probe comprises a fluorophore having a fluorescence property that changes upon hybridization. In some such embodiments the probe comprises a fluorophore and a quencher.

Each of the embodiments summarized above, and described elsewhere herein, can also involve performing some treatment of the subject. For example, in some embodiments the methods summarized above, and described elsewhere herein, may also involve a step of treating the subject with an anti-cancer therapy, such as a chemotherapeutic agent, another therapeutic molecule, with radiation therapy, with surgery, or any combination thereof.

In addition to the various methods described above, and elsewhere herein, in some embodiments the present invention also provides compositions and/or kits comprising any one or more of the primers and probes described herein—i.e. comprising any one or more of SEQ ID NO. 1-92. In certain embodiments, such compositions and/or kits may comprise one or more other components that are useful for and/or compatible with use of such primers and probes, including suitable diluents, buffers, carriers, preservatives, labels, tags, nucleic acid molecules, nucleotides, enzymes (such as polymerase enzymes), and the like. In some embodiments such compositions and/or kits comprise one more positive controls and/or negative controls, such as an HPV16 positive control and/or an HPV16 negative control, an HPV18 positive control and/or an HPV18 negative control, or an HPV33 positive control and/or an HPV33 negative control.

One of skill in the art will recognize that the various embodiments of the present invention described throughout this patent disclosure can be combined in various different ways, and that such combinations are within the scope of the present invention.

One of skill in the art will also recognize that, that the methods and compositions of the present invention described herein are applicable more widely than to only detection of circulating tumor DNA and to only detection in plasma samples, but can also be applied to, and used in conjunction with, detection of various other forms of HPV DNA (i.e. other than ctDNA) and various other tissue samples (i.e. other than plasma), including, but not limited to, blood, urine, cerebrospinal fluid, saliva, and cervical tissue samples. Thus, in each instance in the present specification, and the accompanying claims, in which an embodiment of the invention is described as involving a plasma sample, the present invention also encompasses the analogous embodiment in which another tissue sample (such as blood, urine, cerebrospinal fluid, saliva, or a cervical sample) is used in place of the plasma sample. Similarly, in each instance in the present specification, and the accompanying claims, in which an embodiment of the invention is described as involving ctDNA, the present invention also encompasses the analogous embodiment in which another type of source of HPV DNA (i.e. other than ctDNA) is used or detected.

Similarly, it should also be noted, that each of the methods and compositions of the present invention that are described herein are applicable more widely than to only detection of HPV DNA by PCR, but can also be applied to, and used in conjunction with, any other methods and systems for which the primer and probe sets described herein can be used to amplify, detect, or sequence HPV DNA. Such additional methods include, but are not limited to, amplification-based library generation methods, next generation sequencing methods, and the like. Thus, in each instance in the present specification, and the accompanying claims, in which an embodiment of the invention is described as involving a PCR method, or PCR-based detection, the present invention also encompasses analogous embodiments in which different DNA amplification, detection, and/or sequencing methodologies can be performed using the primer and/or probe sets described.

The invention is further described by the following non-limiting “Examples,” as well as the Figures referred to therein and the descriptions of such Figures provided above.

### Examples

Clinical utility of HPV circulating tumor DNA in the diagnosis and treatment of HPV-associated head and neck cancers

### Summary

We determined the sensitivity of an optimized test for HPV circulating tumor DNA in patients with intact oropharyngeal cancers (HPV+OPSCCs) and in patients with subclinical or minimal residual disease. We prospectively collected plasma specimens before, during, and after treatment. Droplet digital PCR(ddPCR) assays specific for HPV16/33 were used using rationally designed primers/probes. A total of 107 HPV+OPSCC patients receiving definitive chemoradiation (95), surgical resection and adjuvant radiation (9), salvage re-irradiation (1), and palliative radiation (2) were accrued and followed for a median of 14.4 months. In 97 patients with non-metastatic, stage I-IVB OPSCC disease, 90 patients had detectable HPV16 ctDNA and 3 patients had HPV33 ctDNA, indicating an overall sensitivity of 95.6%. No HPV ctDNA was detected in 27 negative controls, indicating 100% specificity. In 3 patients who received salvage or palliative radiation, post-treatment ctDNA predicted disease progression. However, in 3 other cases, no residual HPV16 ctDNA was detected at the conclusion of treatment, yet recurrence was seen at 150, 225, and 330 days later. In 9 patients who underwent upfront surgeries and had pathological indications of residual microscopic disease, in only one patient was HPV16 ctDNA detectable following surgery. The HPV ctDNA compositions and methods developed and used in this study exhibited a very high sensitivity and specificity for intact tumors, demonstrating clinical utility for screening and treatment response monitoring.

### INTRODUCTION

Cancers etiologically driven by human papillomaviruses include squamous cell carcinomas of the oropharynx, cervix, vulva, vagina, anal canal and penis. Pelvic exams and Papanicolaou (Pap) smears are widely adopted screening tools for early detection of early HPV-associated lesions in the cervix; however effective screening approaches for other disease sites are lacking. HPV-associated oropharyngeal cancer (HPV+OPSCC) constitute the largest cohort of those without an available screening paradigm and early detection of these tumors in early stages are more likely to be curable with less treatment such as surgery or radiation alone.

Once diagnosed, HPV+OPSCCs exhibit improved responses to chemoradiotherapy compared to matched HPV negative OPSCCs.^{1,2} For those patients with disease limited to the head and neck with no distant metastatic disease, over 85-90% of patients can be cured with radiation alone, chemoradiotherapy, surgery alone, or a combination of surgery and adjuvant radiation.³ As the toxicity of these therapies is substantial, numerous clinical trials are underway to de-intensify chemoradiation or reduce adjuvant therapies after surgical resection. Thus, accurate biomarkers of treatment response are needed. An emerging category of biomarkers used in clinical trials involve circulating, tumor-derived DNA (ctDNA). Virally-derived ctDNA holds unique advantages over other ctDNA technologies, namely the substrate for detection is viral rather than host, improving the signal to noise ratio, and second, there are often repeated copies of HPV viral DNA within one cancer cell, amplifying ctDNA signal in the blood. ctDNA detection of tumor derived EBV DNA has been successful for early detection of nasopharyngeal cancer(NPC)⁴ and persistent EBV ctDNA levels are a negative predictive factor for recurrence following chemoradiation.⁵⁻⁸ Thus, persistently positive EBV ctDNA is the basis of randomization of adjuvant chemotherapy versus observation following chemoradiation for NPC (clinicaltrials.gov, NCT 02135042).

However, HPV plasma ctDNA studies in head and neck and cervical cancer to date have shown only modest sensitivity in patients with gross disease (19-65%).⁹⁻¹² Further, it is even less clear what is the sensitivity of HPV ctDNA in detecting microscopic disease, such as the patient with only a microscopic positive margin after a surgery or a patient destined to recur locally in the head and neck or at distant metastatic sites months or years after chemoradiation. There is a clinical need for biomarkers to guide adjuvant therapy in these scenarios, but there remain significant questions regarding whether HPV ctDNA can be sensitively measured in HPV+OPSCCs to an extent serviceable for clinical trials.

In the study, we sought to determine the clinical utility of HPV ctDNA using novel rationally designed primers and probes in combination with the highly sensitive droplet digital PCR technology, in patients with macroscopic gross disease and in various clinical settings following initial presentation. We determined the HPV ctDNA kinetics during chemoradiation on a weekly basis. We determined the residual HPV ctDNA level in patients with macroscopic residual disease, such as following palliative RT for recurrent or metastatic disease. Finally, we determined the sensitivity of HPV ctDNA to detect microscopic residual disease by measuring HPV ctDNA levels following surgical outcomes indicating a need for adjuvant radiation. The methods and compositions described herein can be used for HPV ctDNA screening, treatment response monitoring, and minimal residual disease detection after chemoradiation or surgery.

Patients and Methods

This study was conducted at Memorial Sloan Kettering Cancer Center (MSKCC) using banked plasma samples from MSKCC patients consented to IRB-approved biospecimen protocols. Clinical information was retrieved from the medical record under authorization through an IRB-approved retrospective research protocol. Plasma samples were obtained between a period of 2014-2017. Additional negative control plasma samples from normal subjects without cancer were obtained from BioIVT (Westbury, N.Y.), which in turn were collected from consented donors under IRB-approved protocols in the U.S

Study Subjects

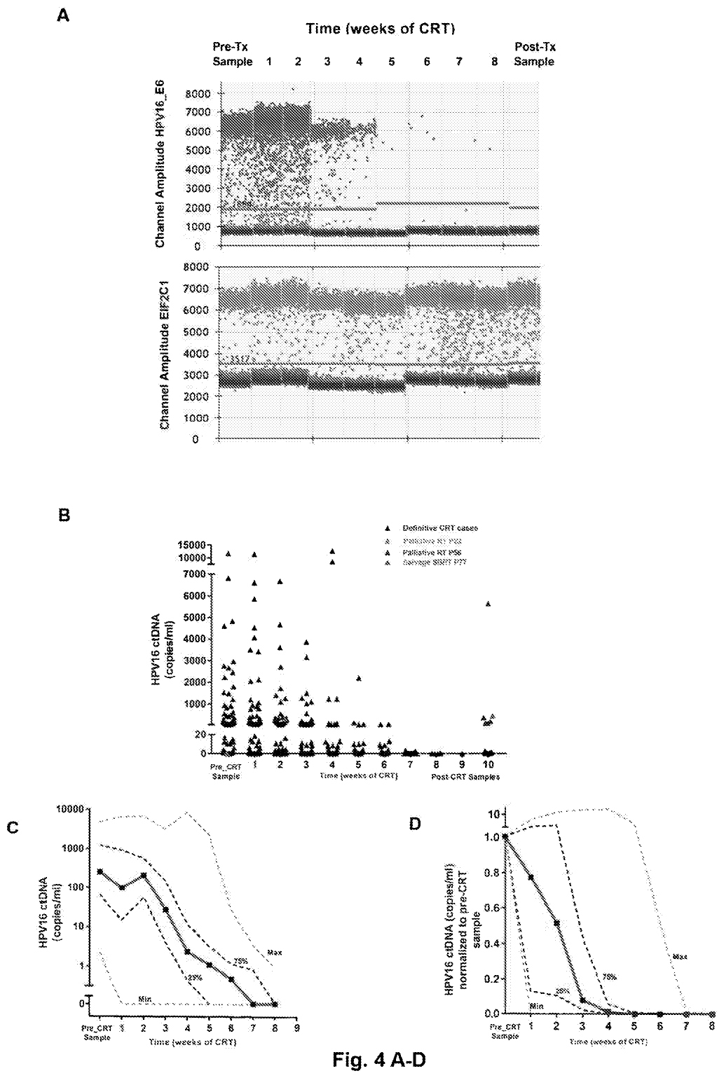

Patients with head and neck and anal cancers who had at least one plasma sample bio-banked were included in this study. A total of 97 patients with stage I-IVB, HPV-positive HNSCCs were included as were 8 patients with anal squamous cell carcinomas. Three additional HPV positive cases with stage IVC OPSCC or recurrent disease were included in FIG. 4. Nine plasma samples were obtained following surgical resection, including 2 patients for whom we had pre-surgical samples available as well. In 97 cases, HPV positivity was defined as p16 overexpression by immunohistochemistry (IHC) with >70% diffuse nuclear/cytoplasmic staining.¹ In 3 additional cases, p16 IHC was not available and there was a clinically reported positive DNA or RNA-based in situ hybridization test for HPV. Samples from 7 patients with HPV-negative head and neck cancers and 20 subjects without cancer were included as negative controls. Information regarding demographics, clinicopathologic features, and outcomes was obtained from the medical record and presented in Table 1 and the results section. Gross tumor volume was determined from the utilized radiation plans for which the treating radiation oncologist contoured gross disease (84 available cases).

Sample Collection and Preparation

10 mL of whole blood was collected from each subject into Cell-free DNA BCT tubes (Streck, Inc.) or BD Vacutainer K2 EDTA tubes (BD Biosciences). Plasma was separated first though centrifugation at 800 g for 5 min, followed by an additional centrifugation at 16,500×g for 5 min. When samples were collected in BD Vacutainer K2-EDTA tubes, plasma was separated within one hour of blood collection and stored at −80C in Eppendorf LoBind tubes (Eppendorf Inc.). When samples were collected into Cell-free DNA BCT tubes, plasma was separated and stored identically at −80C within 48 hours. For repeated samples from the same patient, the same type of tube was used repeatedly. We observed no significant difference in the mean HPV ctDNA level between patient samples collected in each type of tube (not shown). For the negative control samples obtained from BioIVT, blood was collected into BD Vacutainer K2-EDTA tubes and separated as above into plasma within one hour of collection. At a later date, samples were thawed and cfDNA was extracted from 4-5 mL of plasma using Qiagen circulating nucleic acid kits into a 50 mL final elution volume

ctDNA Analysis

For HPV16 positive controls and qPCR standard curve generation, we used the plasmid p1203 PML2d HPV-16 (Addgene plasmid #10869). For HPV33 positive controls, we constructed a HPV33 template plasmid using a gBLOCK (Integrated DNA technologies) ligated BamHI and SaII restriction endonuclease sites in the pUC19 plasmid (Addgene plasmid #50005)

The following HPV16 and HPV33 primers and probes were rationally designed, using the design criteria described herein, and also using Prime3Plus, and ordered through Biorad.

TABLE 4
Sequences

| Primer/Probe | Sequence | SEQ ID NO. |
|---|---|---|
| HPV16 forward | 5′ TATGCACAGAGCTGCAA | SEQ ID NO. 1 |
| primer | ACA 3′ | |
| HPV16 reverse | 5′ GCAAAGTCATATACCTC | SEQ ID NO. 2 |
| primer | ACGTC 3′ | |
| HPV16 probe | 5′ TGTGTGTACTGCAAGCA | SEQ ID NO. 3 |
| | ACAGTTACTG 3′ | |
| | (plus FAM_IowaBlack) | |
| HPV33 forward | 5′ CCACAGTTCGTTTATGT | SEQ ID NO. 4 |
| primer | GTCA 3′ | |
| HPV33 reverse | 5′ TGCCCATAAGTAGTTGC | SEQ ID NO. 5 |
| primer | TGT 3′ | |
| HPV 33 probe | 5′ AGTACAGCAAGTGACCT | SEQ ID NO. 6 |
| | ACGAACCA 3′- | |
| | (plus FAM_IowaBlack) | |
| HPV16 forward | cggtcgatgtatgtcttgtt | SEQ ID NO. 7 |
| (qPCR) | | |
| HPV16 | ctgggtttctctacgtgttc | SEQ ID NO. 8 |
| reverse(qPCR) | | |

For the detection of the PIK3CA_E545K and KRAS_G12D mutations, pre-validated ddPCR mutation assays were used (biorad, assay ID: dHsaCP2000075, dHsaCP2000076 and dHsaCP2000001, dHsaCP2000002).

Cycling conditions were tested to ensure optimal annealing/extension temperature as well as optimal separation of positive from empty droplets. All reactions were performed on a QX200 ddPCR system (Bio-rad). Each sample was evaluated in technical duplicates. PCR reactions contained HPV16, HPV33, PIK3CA_E545K, or KRAS_G12D specific primers and probes, BioRad validated EIF2C1 CNV control primer and probes, DNA and digital PCR Supermix for probes (no dUTP).

For HPV16 and HPV33, reactions were partitioned into a median of ˜15,000 droplets per well using the QX200 droplet generator. Emulsified reactions were amplified on a 96-well thermal cycler using cycling conditions identified during the optimization step (95° C. 10′;40 cycles of 94° C. 30″ 60° C. 1′, 98° C. 10′, 4° C. hold). Plates were read and analyzed with the QuantaSoft software to assess the number of droplets positive for HPV16 or HPV33/EIF2C1 Control, both, or neither. For the detection of PIK3CA_E545K and KRAS_G12D mutations, reactions were partitioned into a median of ˜16,000 droplets per well using the QX200 droplet generator. Emulsified PCRs were run on a 96-well thermal cycler using cycling conditions identified during the optimization step (95° C. 10′;40 cycles of 94° C. 30″ 55° C. 1, 98° C. 10′, 4° C. hold). Plates were read and analyzed with the QuantaSoft software to assess the number of droplets positive for mutant DNA, wild-type DNA, both, or neither. The assay threshold sensitivity was set at 2 mutant droplets.

For HPV16 quantitative PCR, the following primers were used: forward primer 5′ CGGTCGATGTATGTCTTGTT 3′ (SEQ ID NO. 7) and reverse primer 5′ CTGGGTTTCTCTACGTGTTC 3′ (SEQ ID NO. 8). A small amplicon of 55 bp with no probe was used in order to maximize signal from highly fragmented cfDNA. For comparison to ddPCR, the same pool of extracted cfDNA sample was used in both assay systems. The qPCR reactions included 300 nM of each primer, 23 uL of cfDNA (equivalent to 2-2.5 mL of starting plasma), and 25 uL of iQ SYBR green supermix (Bio-rad, Hercules, Calif.). The Bio-rad iQ5 multicolor real-time PCR detection system was used, and a standard curve was generated with the plasmid p1203 PML2d HPV-16.

Pathological Data

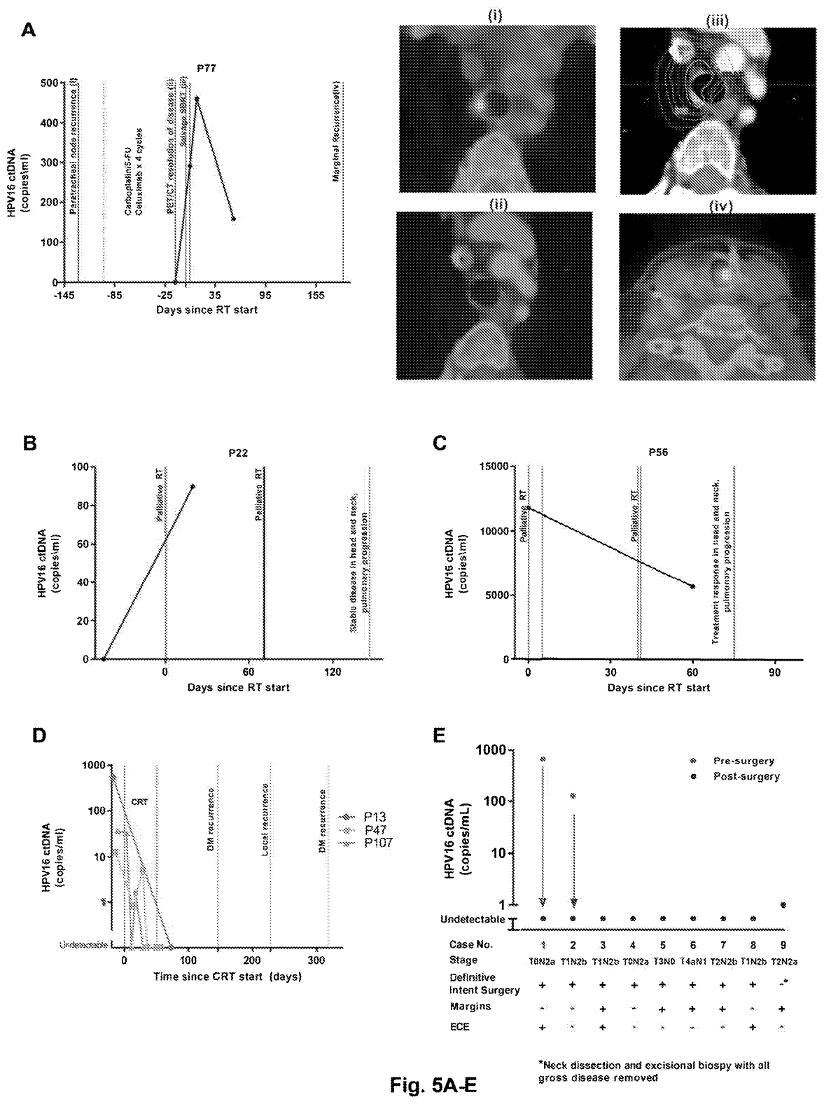

In some cases, targeted next-generation sequencing data of pathological samples were available in the medical record, obtained through the FDA-approved MSK-IMPACT™ sequencing platform. This information was used to apply ddPCR assays for PIK3CA E545K and KRAS G12D, which were previously validated (FIG. 5). To evaluate HPV16 copy number in 10 cases, we extracted genomic DNA from FFPE-preserved pathological samples.

One corresponding hematoxylin and eosin (H&E) stained slide was reviewed by a pathologist and used as a template to scrape, collect, and extract genomic DNA from regions of other unstained slides with >75% tumor cellularity. The genomic DNA was then used for HPV16 ddPCR and the HPV16 copy number was considered as the ratio of HPV16 positive droplets to droplets containing the reference gene EIF2C1.

Statistical Analysis

Sensitivity for the stage I-IVB cases was defined as the number of pre-treatment cfDNA samples that were positive for HPV ctDNA divided by the total number of cases (N=97). Specificity was defined as the number of negative control samples that had zero HPV+droplets, divided by the total number of negative controls (N=27). To evaluate for any correlation between gross tumor volume or copy number with the initial ctDNA levels, Pearson's correlation coefficient was calculated with GraphPad prism software

Results

Patient Characteristics

To assess the clinical utility of HPV ctDNA tests, a series of plasma specimens were collected and stored prospectively from 2014-2017 under optimal conditions for ctDNA studies, using either cell-free DNA BCT tubes (Streck, Inc.) for preservation of ctDNA or with BD Vacutainer K2 EDTA tubes (BD Biosciences) and immediate processing. The series included 97 patients at their initial diagnosis of stage I-IVB HPV+OPSCC and 9 patients following surgical resection of disease (Table 1). All patients were fully staged through physical examination and PET/CT to rule out metastatic disease. Three additional cases of recurrent HPV+OPSCC disease or unbiopsied but suspected metastatic, stage IVC disease were included. In 104 cases, patients were clinically considered to be HPV+through detection of p16 overexpression by IHC in tumor biopsy specimens. In 3 cases p16 IHC was not performed but there was a positive, clinically reported DNA or RNA ISH test for high risk HPV strains.

The clinical characteristics of patients in the series is similar to previously published cohorts from our institution¹³⁻¹⁵ with stage IVA, T2 and N2b disease as the most common AJCC 7 stage, T stage and N stage, respectively. In 95 cases, patients received chemoradiation as definitive therapy. In 1 case, transoral robotic surgery followed by adjuvant radiation was employed and 1 case transoral robotic surgery alone was used. In 7 additional cases, post-surgical samples were collected to evaluate the ability of HPV ctDNA by ddPCR to detect likely microscopic residual disease, such as in the case of a positive margins or extracapsular extension.

Assay Design

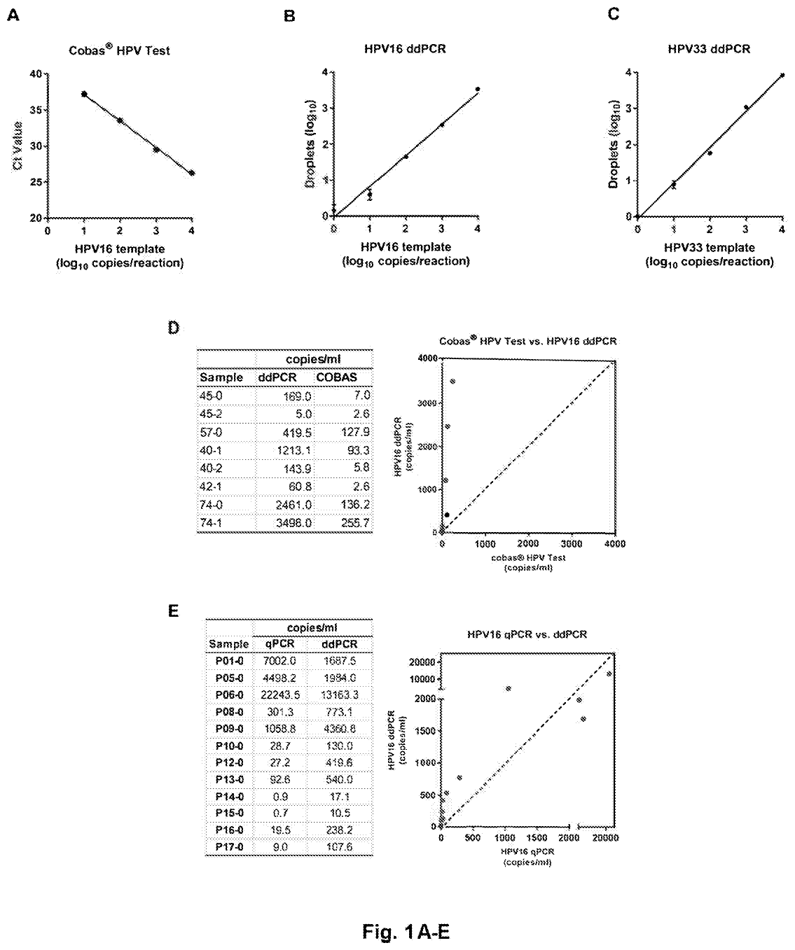

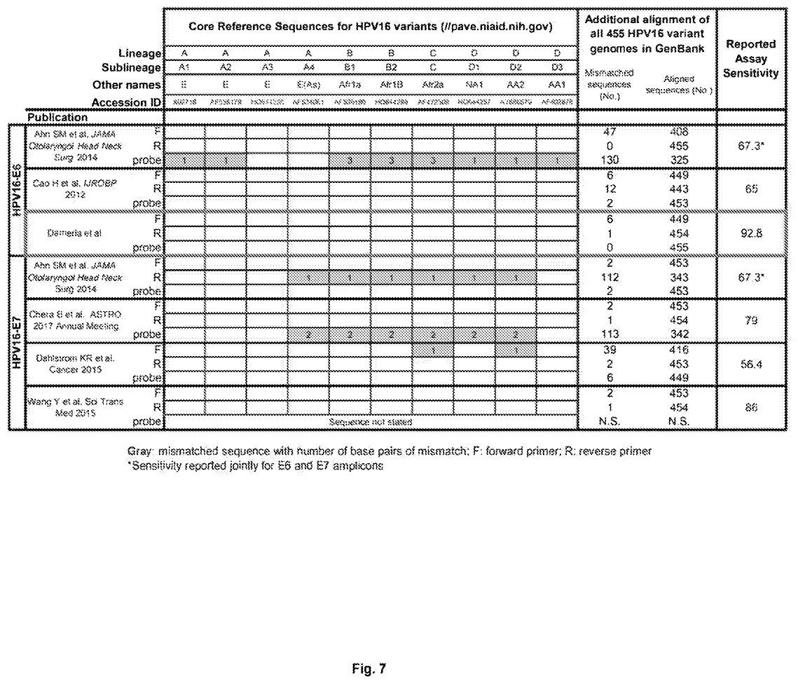

Droplet digital PCR has emerged as a preferred ctDNA detection strategy¹⁶ due to the high sensitivity and ability to quantify absolute numbers of DNAs without standard samples and curves that would need to be cross-standardized across clinical laboratories as was required for EBV ctDNA in NPC.¹⁷ We first sought to develop the best possible primer-probe set for droplet digital PCR using three criteria: 1) location within the E6 or E7 oncogenes as these sequences are the most highly amplified sequences in tumor genomes¹⁸, 2) amplicon size, with smaller amplicons considered to be better in view of the highly fragmented nature of cfDNA, and 3) primers/probes that perfectly match both European and non-European HPV16 isolates, maximizing universality of the test. We utilized the Papillomavirus Episteme resource, sponsored by the U.S. National Institute of Allergy and Infectious Diseases, and identified the 10 sequences representative of HPV16 sublineages (A1-A4, B1-B2, C1, D1-3), spanning European, Asian, and African isolates of HPV16 (FIG. 7). We chose primers and probes within E6 and E7 that match these 10 most common sequences exactly and then subsequently compared these sequences to an additional 455 HPV16 isolates found in GenBank. A primer/probe set in E6 with a length of 97 bp met all criteria and demonstrated high PCR efficiency and sensitivity down to a single molecule of template HPV16 DNA in a droplet digital PCR assay (FIG. 1B).

We then compared the performance of ddPCR to two additional HPV ctDNA detection platforms, the Roche Cobas® HPV Test, and a quantitative PCR assay. The clinically available Roche Cobas® HPV Test is based upon real-time PCR technology and is designed for Pap smears. It detects a panel of high risk HPV species and also includes a reflex test for HPV16/18. We used a serial dilution of HPV16 template on a plasmid and generated a standard curve to render the assay quantitative (FIG. 1A). We then compared levels of HPV16 ctDNA in 8 patient samples as measured by ddPCR and by the Roche Cobas® HPV Test in the same samples and found ddPCR to detect a higher number of copies per mL of plasma (p=0.008, Wilcoxon matched-pairs signed rank test) while ctDNA levels detected by both methods are highly correlated (r²=0.82, p=0.002). The signal in paired samples was a median of 15.9-fold higher with ddPCR. While the specific Roche Cobas® primer/probe sequences are proprietary, the amplicon length is publicized as ˜200 bp, likely too large for fragmented ctDNA which has a median size of 176 bp.¹⁶

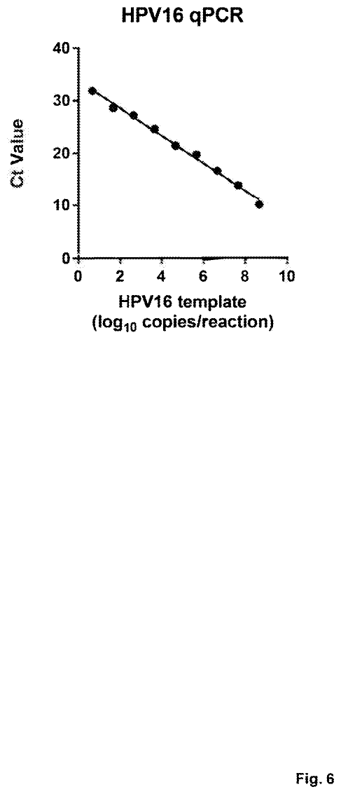

We then compared ddPCR to a quantitative PCR assay wherein the amplicon size is highly minimized through omission of a probe altogether. We chose a 55 bp qPCR amplicon in E7, developed standard curves (FIG. 6), and compared levels of HPV16 ctDNA in 12 samples by ddPCR and qPCR. We found ddPCR to be more sensitive at lower ctDNA values (arbitrary cutoff. <1000 HPV ctDNA copies/mL) by Wilcoxon matched-pairs signed rank test (p=0.008) though each approach was highly correlated (r²=0.88, p<0.0001). Amongst all paired samples, HPV ctDNA was a median of 5.2-fold higher with ddPCR. At very high levels of HPV16, the HPV ctDNA signal can begin to saturate, as observed in 3 samples because only 20,000 droplets are read on the Biorad QX200 platform. The higher sensitivity of the ddPCR assay despite a larger amplicon size may be secondary to the partitioning of DNAs into droplets and reduced inhibitory factors compared to qPCR.¹⁹ Thus, we elected to use the ddPCR assay for all remaining patient samples.

The bioinformatic algorithm developed and used to select primers and probes for HPV16 in these studies can be summarized as follows: Step 1—the 10 most representative genomes of HPV variants (available at pave.niaid.nih.giv) were identified (accession numbers K02718, AF536179, HQ644236, AF534061, AF536180, HQ644257, AY686579, AF402678). Step 2—Alignment with Clustalw algorithm (muscle 3.8). Step 3—Limited selection to early genes E5, E6 and E7 of the HPV16 genome. Step 4-3 amplicons in E5, E6, and E7 were chosen. Step 5. Cross-referenced with 445 HPV16 variants from NCBI Genbank alignment. Step 6—Blast against the human genome and other HPV strains. Step 7—E6 assay showed best ddPCR performance. Step 8—forward and reverse primer and probe selected. Step 8. Standard curves and testing patient plasma samples.

Sensitivity and Specificity of HPV16 and HPV33 ddPC

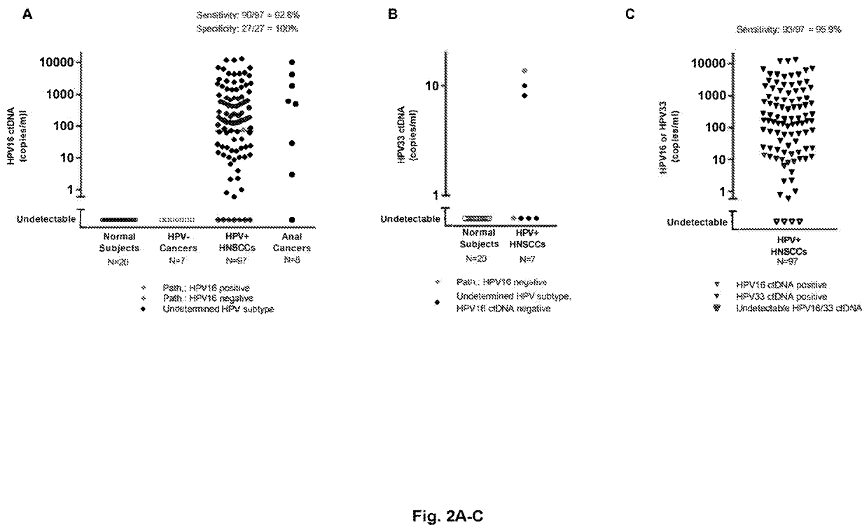

In 97 stage I-IVB HPV+OPSCC patients with pre-treatment cfDNA, HPV16 ctDNA was detected in 90 patients, demonstrating a sensitivity of 92.8%. We also tested 20 samples from normal subjects without cancer and 7 samples from patients with HPV negative head and neck cancers and no droplets were detected in these 27 cases, demonstrating 100% specificity. In four cases, the patient's tumor biopsy sample had been used to specifically identify the HPV subtype: in 3 cases through RNA ISH with HPV16 specific probes and in one case by HPV16 specific RT-PCR. In 2 of those 4 cases, the tumor pathology was HPV16 negative and in both cases no HPV16 ctDNA was detected (FIG. 2A). In the 2 other cases, the tumor pathology was HPV16 positive and in both of these cases, HPV16 ctDNA was readily detected in plasma (FIG. 2A), demonstrating 100% concordance (4/4) between plasma and the tumor specimen in HPV subtyping. The mean HPV16 ctDNA level was 1218 copies/mL (range 0-13,163) with no clear cutpoints for classifications into groups. We also tested 8 plasma samples from patients with anal cancers and detected HPV16 ctDNA with a mean of 2151 copies/mL in 7, demonstrating the ddPCR assay can be employed in other HPV+malignancies (FIG. 2A)

Aside from HPV16, the next most common HPV type associated with OPSCC is HPV33.²⁰ We developed a ddPCR assay for this strain specifically and found a high degree of linearity of signal (FIG. 1C) and no HPV33 positive droplets in 20 samples from normal subjects without cancer. We then tested the 7 cfDNA samples which were negative for HPV16 ctDNA by ddPCR and found that three of these samples were HPV33 positive at 8.1, 13.6, and 10.0 copies/mL (FIG. 2B). Thus, the overall sensitivity for HPV16 or HPV33 ctDNA was detectable in 93 of 97 patients, with 4 samples remaining undetectable (95.9% sensitivity)

Predictors of HPV16 ctDNA Level

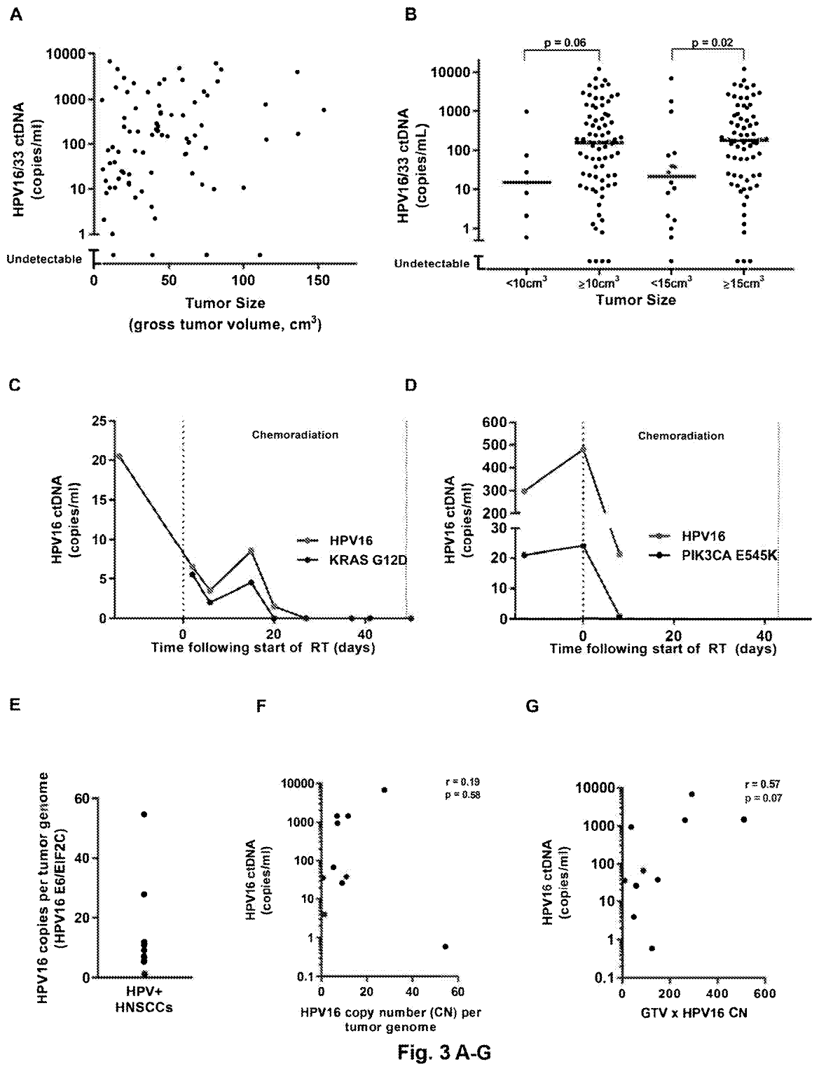

We then sought to determine factors that influence ctDNA levels. Ninety five of the 97 patients in this cohort underwent definitive chemoradiation and the volume of gross disease was carefully outlined by the treating radiation oncologist. However, the volume of gross disease, termed gross tumor volume (GTV), did not correlate with HPV16 ctDNA levels overall (FIG. 3A). We did find that small tumors, while readily detectable, exhibited lower median HPV ctDNA signals (FIG. 3B). In tumors less than 10 cm³, 7 of 7 were detectable and the median HPV16 level was 15 vs. 157 copies/mL for tumors larger than 10 cm³ (p=0.06, Mann Whitney u test). In tumors less than 15 cm³, 15 of 16 were detectable with a median HPV ctDNA signal of 20.9 vs. 177.4 copies/mL in tumors larger than 15 cm³ (p=0.02, Mann Whitney u test)

In two cases (1 HPV+OPSCC and 1 anal SCC), we compared HPV16 ctDNA levels with ctDNA levels of another driver mutation. In a T1N1 anal SCC case, the HPV16 ctDNA levels tracked with PIK3CA E545K levels before and during chemoradiotherapy, but HPV16 ctDNA was generally 14-fold to 21-fold higher in magnitude (FIG. 3C). In a T1N2b HPV+OPSCC case, HPV16 ctDNA also tracked along with KRAS G12D levels in the plasma, before, during and after radiotherapy, but in this case HPV16 ctDNA levels were only 1.2-1.9-fold higher in magnitude (FIG. 3D). These two cases suggest that the copy number of HPV16 in the tumor can impact that levels of HPV16 ctDNA in the plasma

Since the number of HPV genomic copies per tumor genome can be widely variable, we determined the HPV copy number directly in 10 cases, which ranged from 0.8 to 57 copies per tumor genome (FIG. 3E). We then asked whether the HPV16 copy number correlates with HPV16 ctDNA level as a single covariate and again found very little correlation (FIG. 3F). Since tumor volume is proportional to the number of cancer cells and the HPV16 copy number per cancer cell was known in 10 cases, we postulated that the product of tumor volume (GTV) and HPV16 copy number would be proportional to HPV16 ctDNA and found a modest, nonsignificant correlation (r=0.57, p=0.07), indicating that other unspecified factors are also important (FIG. 3G). For instance, in other reports, the degree of tumor necrosis can influence ctDNA shedding.²¹

HPV ctDNA Response Kinetics During Chemoradiation

In a subset of patients, we obtained weekly samples during and after chemoradiation for stage I-IVB disease and in all cases observed rapid declines. FIG. 4A shows the raw data of one patient over the course of radiotherapy and the number of droplets positive for HPV16 (blue) drops rapidly while the number of droplets positive for a reference gene (EIF2C1) remains unchanged. When considering all 456 samples in 68 patients with multiple samples, HPV16 ctDNA was generally cleared by week 7 following the start of chemoradiation (FIG. 4B), with the exception of 3 patients in which HPV16 ctDNA levels remained detectable at 10 weeks. These three patients have not recurred clinically with follow up of 15.1, 15.3, and 20.6 months. In 26 patients in which a complete set of weekly samples were available, the median, 25-75% quartiles, minimum and maximum levels (FIG. 4C) find a high degree of heterogeneity in response kinetics. Interestingly, in 10 of 26 cases, we observed an increase in HPV16 ctDNA between the first and second week of chemoradiation, possibly reflecting an initial surge of cell death. From week 2 through week 7, median levels declined rapidly. When response kinetics were normalized same-patient pre-treatment levels (FIG. 4D), the response kinetics are steeper than the raw values (FIG. 4C), reflecting the that week of clearance of HPV16 ctDNA can be influenced by the quantity of original pre-treatment HPV16 ctDNA

Microscopic Residual Disease Following Chemoradiation

In addition to the 97 stage I-IVB HPV+OPSCC, we also obtained samples in three patients with recurrent or early stage IVC disease. In case P77 (FIG. 5A), a patient had received previous definitive chemoradiation, developed a paratracheal node recurrence 15 months later, which was the only site of disease identified on staging PET/CT (FIG. 5A, panel i). The patient received initial chemotherapy (carboplatin/5-FU/cetuximab) and achieved resolution of disease by PET/CT after 4 cycles (FIG. 5A, panel ii). The patient received salvage stereotactic body radiotherapy (SBRT, 27 Gy in 3 fractions) to the paratracheal node (FIG. 5A, panel iii), but HPV16 ctDNA remained elevated 60 days following SBRT and a recurrence was detected 121 days later (FIG. 5A, panel iv). In 2 cases, (P56 and P22), patients presented with highly suspicious but biopsied pulmonary metastases synchronously with the primary disease in the head and neck. In both cases, a palliative radiation regimen was employed, involving cyclical rounds of short courses of radiation. In both cases, HPV16 ctDNA remained elevated despite palliative radiation and both patients' pulmonary disease progressed 70 and 135 days later, respectively. These three cases illustrate that in some cases, persistent HPV cfDNA can forewarn local and distance recurrences and progression of disease. However, in our dataset, three were also three patients with stage I-IVB disease with late recurrences at 146, 228, and 318 days following the start of radiation whereas immediate post-treatment HPV ctDNA samples failed to detect residual disease (FIG. 5D).

Microscopic Residual Disease Following Surgery

We then asked whether HPV ctDNA could detect microscopic, minimal residual disease following transoral robotic surgery (TORS). Following surgery, the pathologic sample is reviewed for features indicating the need for adjuvant radiotherapy because of a high risk of microscopic disease. These include positive surgical margins, extracapsular extension, and multiple positive lymph nodes. In 8 cases, a surgery with definitive intent was performed, and risk factors for recurrence included positive margins (4 cases), extracapsular extension (3 cases), and multiple positive lymph nodes (4 cases). In none of these cases was residual HPV16 ctDNA detected. In an additional case of a patient with T2N2a disease, a neck dissection was conducted for N2a disease, but only an excisional biopsy of a base of tongue lesion was performed, leaving no gross residual disease. In only this case was residual HPV16 ctDNA at low levels

Discussion

In this study, we have demonstrated that HPV ctDNA can be detected in nearly all patients with HPV+OPSCC, a marked improvement over previous reports and likely due to four primary improvements: A) use of the droplet digital PCR technique which we found to be superior to qPCR (FIG. 1), B) design of HPV16 primers/probes that match all HPV16 variants (FIG. 7), and the other primer/probe design criteria described above, C) use of a combination of HPV16 and HPV33 tests (FIG. 2), and D) plasma collection and storage procedures optimized for cfDNA. The overall sensitivity was 95.9% in our cohort, a figure similar to the HPV subtype distribution for HPV16 and 33 in HPV+OPSCC. In the 73 HPV+cases characterized by The Cancer Genome Atlas project, 61 were HPV16+ and 8 were HPV33+, a total of 94.5% of cases. We demonstrated that the assay was portable to other HPV associated malignancies as we could also detect HPV ctDNA in 7 of 8 anal squamous cell carcinomas and demonstrated concordance between HPV strain subtyping in the tumor specimen and in the plasma. The assay could readily detect small tumors as HPV ctDNA was measured in 8 of 8 cases with primary tumors less than 10 cm³. Thus, HPV ctDNA by ddPCR has the sensitivity and characteristics needed for screening for early HPV-associated cancers.

In intra-patient comparisons, we find that HPV ctDNA levels are directly related to tumor volume and burden because levels declined rapidly for all patients receiving definitive chemoradiation as their primary tumors decreased in size (FIG. 4). Thus, HPV ctDNA could be readily used in clinical trials evaluating systemic agents to assess a patient's change in overall disease burden. HPV ctDNA by ddPCR has particular advantages over other ctDNA technologies, such as next generation sequencing, as there is no competing normal genomic DNA and thus HPV ctDNA is expressed as an absolute level rather than a ratio or percentage of all DNAs (FIG. 1B-C).

In inter-patient comparisons, however, initial HPV ctDNA levels are heterogeneous as there was no correlation between gross tumor volume and ctDNA levels across our patient cohort. We propose that ctDNA levels are a function of tumor size and HPV copy number per genome (FIG. 3G), but additional unknown tumor-specific factors are also important.

While persistent HPV ctDNA levels following treatment could be detected in several recurrent or palliative disease settings (FIG. 5), the ctDNA levels after conclusion of chemoradiation failed to detect late recurrences in three cases. In addition, there were three cases with persistent HPV ctDNA at conclusion of chemoradiation at week 10 yet have not recurred with 15-20 months of follow-up. These persistent cases may represent false-positives, or they may also reflect the pattern of disease regression in HPV positive OPSCC.

In the era when neck dissections following chemoradiation were routine, persistent disease was found in the pathological sample in 20% of cases 6-8 weeks following completion of radiation; however, recent data also note that many if not most neck dissections are unnecessary. Thus, slow regression of disease that continues well after radiation is completed is a known phenomenon.

While HPV ctDNA can be detected in 95.6% of cases with intact disease, its ability to detect true minimal residual disease is less clear. A tumor 1 cm³ in size, approximately the size of radiographic detection of a lymph node, is estimated to contain 10⁸-10⁹ cells²² whereas in principle a single residual clonogen following surgery or chemoradiation can recur months or years later. Thus, there are 8-9 or more logs of tumor cells in patients with intact disease, but there is only ˜3 logs of dynamic range in HPV ctDNA signal (median initial value is 1218 copies/mL). Thus, in 9 cases with pathological indications of minimal residual disease following surgery, such as a positive margin or extracapsular extension, HPV ctDNA was only detectable in one case in which an excisional biopsy rather than a definitive resection was performed (FIG. 5).

All samples on this study were collected prospectively, though not every patient was willing to provide weekly samples during chemoradiation, thus leading to heterogeneity in available data points. Post-treatment samples were obtained following completion of treatment in weeks 8-10. Later time points may have provided additional information.

Our results demonstrate that HPV ctDNA can be effectively used with very high sensitivity and specificity for intact tumors, demonstrating clinical utility for screening and treatment response monitoring.

### REFERENCE LIST

1 Ang, K. K. et al. Human papillomavirus and survival of patients with oropharyngeal cancer. N Engl J Med 363, 24-35, doi:10.1056/NEJMoa0912217 (2010).
2 Fakhry, C. et al. Improved survival of patients with human papillomavirus-positive head and neck squamous cell carcinoma in a prospective clinical trial. J Natl Cancer Inst 100, 261-269, doi:10.1093/jnci/djn011 (2008).
3 Wang, M. B., Liu, I. Y., Gornbein, J. A. & Nguyen, C. T. HPV-Positive Oropharyngeal Carcinoma: A Systematic Review of Treatment and Prognosis. Otolaryngol Head Neck Surg 153, 758-769, doi:10.1177/0194599815592157 (2015).
4 Chan, K. C. A. et al. Analysis of Plasma Epstein-Barr Virus DNA to Screen for Nasopharyngeal Cancer. N Engl J Med 377, 513-522, doi:10.1056/NEJMoa1701717 (2017).
Lin, J. C. et al. Quantification of plasma Epstein-Barr virus DNA in patients with advanced nasopharyngeal carcinoma. N Engl J Med 350, 2461-2470, doi:10.1056/NEJMoa032260 (2004).
6 Hong, R. L., Lin, C. Y., Ting, L. L., Ko, J. Y. & Hsu, M. M. Comparison of clinical and molecular surveillance in patients with advanced nasopharyngeal carcinoma after primary therapy: the potential role of quantitative analysis of circulating Epstein-Barr virus DNA. Cancer 100, 1429-1437, doi:10.1002/cncr.20129 (2004).
7 Chan, A. T. et al. Concurrent chemotherapy-radiotherapy compared with radiotherapy alone in locoregionally advanced nasopharyngeal carcinoma: progression-free survival analysis of a phase III randomized trial. J Clin Oncol 20, 2038-2044, doi:10.1200/JCO.2002.08.149 (2002).
8 Wang, W. Y. et al. Long-term survival analysis of nasopharyngeal carcinoma by plasma Epstein-Barr virus DNA levels. Cancer 119, 963-970, doi:10.1002/cncr.27853 (2013).
9 Ahn, S. M. et al. Saliva and plasma quantitative polymerase chain reaction-based detection and surveillance of human papillomavirus-related head and neck cancer. JAMA Otolaryngol Head Neck Surg 140, 846-854, doi:10.1001/jamaoto.2014.1338 (2014).
10 Cao, H. et al. Quantitation of human papillomavirus DNA in plasma of oropharyngeal carcinoma patients. Int J Radiat Oncol Biol Phys 82, e351-358, doi:10.1016/j.ijrobp.2011.05.061 (2012).
11 Mazurek, A. M., Rutkowski, T., Fiszer-Kierzkowska, A., Malusecka, E. & Skladowski, K. Assessment of the total cfDNA and HPV16/18 detection in plasma samples of head and neck squamous cell carcinoma patients. Oral Oncol 54, 36-41, doi:10.1016/j.oraloncology.2015.12.002 (2016).
12 Wang, Y. et al. Detection of somatic mutations and HPV in the saliva and plasma of patients with head and neck squamous cell carcinomas. Sci Transl Med 7, 293ra104, doi:10.1126/scitranslmed.aaa8507 (2015).
13 Leeman, J. E. et al. Patterns of Treatment Failure and Postrecurrence Outcomes Among Patients With Locally Advanced Head and Neck Squamous Cell Carcinoma After Chemoradiotherapy Using Modern Radiation Techniques. JAMA Oncol 3, 1487-1494, doi:10.1001/jamaoncol.2017.0973 (2017).
14 Zumsteg, Z. S. et al. The toxicity and efficacy of concomitant chemoradiotherapy in patients aged 70 years and older with oropharyngeal carcinoma in the intensity-modulated radiotherapy era. Cancer 123, 1345-1353, doi:10.1002/cncr.30495 (2017).
15 Setton, J. et al. Intensity-modulated radiotherapy in the treatment of oropharyngeal cancer: an update of the Memorial Sloan-Kettering Cancer Center experience. Int J Radiat Oncol Biol Phys 82, 291-298, doi:10.1016/j.ijrobp.2010.10.041 (2012).
16 Diaz, L. A., Jr. & Bardelli, A. Liquid biopsies: genotyping circulating tumor DNA. J Clin Oncol 32, 579-586, doi:10.1200/JCO.2012.45.2011 (2014)
17 Le, Q. T. et al. An international collaboration to harmonize the quantitative plasma Epstein-Barr virus DNA assay for future biomarker-guided trials in nasopharyngeal carcinoma. Clin Cancer Res 19, 2208-2215, doi:10.1158/1078-0432.CCR-12-3702 (2013).
18 Akagi, K. et al. Genome-wide analysis of HPV integration in human cancers reveals recurrent, focal genomic instability. Genome Res 24, 185-199, doi:10.1101/gr.164806.113 (2014).
19 Dingle, T. C., Sedlak, R. H., Cook, L. & Jerome, K. R. Tolerance of droplet-digital PCR vs real-time quantitative PCR to inhibitory substances. Clin Chem 59, 1670-1672, doi:10.1373/clinchem.2013.211045 (2013).
20 Cancer Genome Atlas, N. Comprehensive genomic characterization of head and neck squamous cell carcinomas. Nature 517, 576-582, doi:10.1038/nature14129 (2015).
21 Abbosh, C. et al. Phylogenetic ctDNA analysis depicts early-stage lung cancer evolution. Nature 545, 446-451, doi:10.1038/nature22364 (2017).
22 Del Monte, U. Does the cell number 10(9) still really fit one gram of tumor tissue? Cell Cycle 8, 505-506, doi:10.4161/cc.8.3.7608 (2009).

We claim:

1. A method of detecting HPV circulating tumor DNA in a plasma sample from a subject, the method comprising:
(a) performing a polymerase chain reaction comprising contacting a plasma sample obtained from a subject, or DNA obtained therefrom, with a primer pair consisting of a forward primer and a reverse primer, wherein the forward and reverse primer:
a. each bind to a target sequence in the HPV genome that:
i. is within the E6 and/or E7 region of the HPV genome, and
ii. is 100% conserved between European and non-European HPV isolates, and
b. together span a region in the HPV genome that is about 175 nucleotides in length, or less, and
(b) determining the presence of a specific amplified PCR product generated in the polymerase chain reaction,
wherein if a specific amplified PCR product is detected, the plasma sample contains HPV circulating tumor DNA.

2. The method claim 1, wherein the presence of a specific amplified PCR product indicates that the subject has an HPV-associate malignancy.

3. The method claim 1, wherein step (b) further comprises determining the quantity of a specific amplified PCR product generated in the polymerase chain reaction, and wherein the quantity of the specific amplified PCR product correlates to tumor burden of the HPV-positive tumor in the subject.

4. A method of monitoring progression, recurrence, or response to therapy of an HPV-positive tumor in a subject, the method comprising performing the method of claim 1 using both a first plasma sample obtained from a subject at a first time point and a second plasma sample obtained from a subject at a second time point, or DNA obtained from such a plasma sample, and determining the quantity of a specific amplified PCR product generated in the polymerase chain reaction in both the first plasma sample and the second plasma sample, wherein an increase in the quantity of the specific amplified PCR product in the second sample as compared to the first sample indicates an increase in HPV-positive squamous cell carcinoma burden from the first time point to the second time point, and a decrease in the quantity of the specific amplified PCR product in the second sample as compared to the first sample indicates a decrease in HPV-positive squamous cell carcinoma burden from the first time point to the second time point.

5. The method of claim 1, wherein the HPV circulating tumor DNA is from a squamous cell carcinoma of the head and neck, oropharynx, cervix, vulva, vagina, anal canal or penis.

6. The method of claim 1, wherein the subject has, or is suspected of having, an HPV-associated malignancy/tumor that is a squamous cell carcinoma of the head and neck, oropharynx, cervix, vulva, vagina, anal canal or penis.

7. The method of claim 1, wherein the HPV circulating tumor DNA is DNA from HPV type 16, 18, 31, 33, 35, 39, 45, 51, 52, 56, 58 or 59.

8. The method of claim 1, wherein the HPV circulating tumor DNA is HPV16 DNA.

9. The method of claim 1, wherein the HPV circulating tumor DNA is HPV16 DNA and wherein the subject has, or is suspected of having, a squamous cell carcinoma of the oropharynx or of the anal canal.

10. The method of any of claim 1, wherein the HPV DNA is HPV16 DNA, and wherein the primer pair is selected from the group consisting of:
(a) a forward primer comprising SEQ ID NO. 1 and a reverse primer comprising SEQ ID NO. 2,
(b) a forward primer comprising SEQ ID NO. 9 and a reverse primer comprising SEQ ID NO. 10,
(c) a forward primer comprising SEQ ID NO. 11 and a reverse primer comprising SEQ ID NO. 12,
(d) a forward primer comprising SEQ ID NO. 13 and a reverse primer comprising SEQ ID NO. 14,
(e) a forward primer comprising SEQ ID NO. 15 and a reverse primer comprising SEQ ID NO. 16,
(f) a forward primer comprising SEQ ID NO. 17 and a reverse primer comprising SEQ ID NO. 18,
(g) a forward primer comprising SEQ ID NO. 19 and a reverse primer comprising SEQ ID NO. 20,
(h) a forward primer comprising SEQ ID NO. 21 and a reverse primer comprising SEQ ID NO. 22,
(i) a forward primer comprising SEQ ID NO. 23 and a reverse primer comprising SEQ ID NO. 24, and
(j) a forward primer comprising SEQ ID NO. 25 and a reverse primer comprising SEQ ID NO. 26.

11. The method of claim 1, wherein the HPV DNA is HPV33 DNA.

12. The method of any of claim 1, wherein the HPV DNA is HPV33 DNA and wherein the subject has a squamous cell carcinoma of the oropharynx or of the anal canal.

13. The method of claim 1, wherein the HPV is HPV33, and wherein the primer pair is selected from the group consisting of:
(a) a forward primer comprising SEQ ID NO. 4 and a reverse primer comprising SEQ ID NO. 5,
(b) a forward primer comprising SEQ ID NO. 36 and a reverse primer comprising SEQ ID NO. 37,
(c) a forward primer comprising SEQ ID NO. 38 and a reverse primer comprising SEQ ID NO. 39,
(d) a forward primer comprising SEQ ID NO. 40 and a reverse primer comprising SEQ ID NO. 41,
(e) a forward primer comprising SEQ ID NO. 42 and a reverse primer comprising SEQ ID NO. 43,
(f) a forward primer comprising SEQ ID NO. 44 and a reverse primer comprising SEQ ID NO. 45,
(g) a forward primer comprising SEQ ID NO. 46 and a reverse primer comprising SEQ ID NO. 47,
(h) a forward primer comprising SEQ ID NO. 48 and a reverse primer comprising SEQ ID NO. 49,
(i) a forward primer comprising SEQ ID NO. 50 and a reverse primer comprising SEQ ID NO. 51,
(j) a forward primer comprising SEQ ID NO. 52 and a reverse primer comprising SEQ ID NO. 53.

14. The method of claim 1, wherein the HPV DNA is HPV18 DNA.

15. The method of claim 1, wherein the HPV DNA is HPV18 DNA and wherein the subject has a squamous cell carcinoma of the head and neck, oropharynx, cervix, vulva, vagina, anal canal or penis.

16. The method of claim 1, wherein the HPV is HPV18, and wherein the primer pair is selected from the group consisting of:
(a) a forward primer comprising SEQ ID NO. 63 and a reverse primer comprising SEQ ID NO. 64,
(b) a forward primer comprising SEQ ID NO. 65 and a reverse primer comprising SEQ ID NO. 66,
(c) a forward primer comprising SEQ ID NO. 67 and a reverse primer comprising SEQ ID NO. 68,
(d) a forward primer comprising SEQ ID NO. 69 and a reverse primer comprising SEQ ID NO. 70,
(e) a forward primer comprising SEQ ID NO. 71 and a reverse primer comprising SEQ ID NO. 72,
(f) a forward primer comprising SEQ ID NO. 73 and a reverse primer comprising SEQ ID NO. 74,
(g) a forward primer comprising SEQ ID NO. 75 and a reverse primer comprising SEQ ID NO. 76,
(h) a forward primer comprising SEQ ID NO. 77 and a reverse primer comprising SEQ ID NO. 78,
(i) a forward primer comprising SEQ ID NO. 79 and a reverse primer comprising SEQ ID NO. 80,
(j) a forward primer comprising SEQ ID NO. 81 and a reverse primer comprising SEQ ID NO. 82.

17. The method of claim 1, comprising performing the polymerase chain reaction using both a first primer pair and a second primer pair, or performing a first polymerase chain reaction using a first primer pair and a second polymerase chain reaction using a second primer pair, wherein both the first and second primer pairs consist of a forward primer and a reverse primer, wherein the forward and reverse primer:
a. each bind to a target sequence in the HPV genome that:
i. is within the E6 and/or E7 region of the HPV genome, and
ii. is 100% conserved between European and non-European HPV isolates, and
b. together span a region in the HPV genome that is about 175 nucleotides in length, or less,
and wherein either (a) the first primer pair is specific for HPV16 and the second primer pair is specific for HPV33, (b) the first primer pair is specific for HPV16 and the second primer pair is specific for HPV18, or (c) the first primer pair is specific for HPV18 and the second primer pair is specific for HPV33.

18. The method of claim 1, comprising performing the polymerase chain reaction using a first primer pair, a second primer pair, and a third primer pair, or performing a first polymerase chain reaction using a first primer pair, a second polymerase chain reaction using a second primer pair and a third polymerase chain reaction using a third primer pair wherein each of the primer pairs consist of a forward primer and a reverse primer, wherein the forward and reverse primer:
a. each bind to a target sequence in the HPV genome that:
i. is within the E6 and/or E7 region of the HPV genome, and
ii. is 100% conserved between European and non-European HPV isolates, and
b. together span a region in the HPV genome that is about 175 nucleotides in length, or less,
and wherein the first primer pair is specific for HPV16, the second primer pair is specific for HPV33, and the third primer pair is specific for HPV18.

19. The method of any of claims 16-18, wherein the HPV16 primer pair is selected from the group of primers listed in claim 10, the HPV18 primer pair is selected from the group of primers listed in claim 13, and the HPV33 primer pair is selected from the group of primers listed in claim 16.

20. The method of claim 1, wherein the forward primer and the reverse primer span a region in the E6 and/or E7 region of the HPV genome that is about 150 nucleotides in length, or less.

21. The method of claim 1, wherein the forward primer and the reverse primer span a region in the E6 and/or E7 region of the HPV genome that is about 125 nucleotides in length, or less.

22. The method of claim 1, wherein the forward primer and the reverse primer span a region in the E6 and/or E7 region of the HPV genome that is about 100 nucleotides in length, or less.

23. The method of claim 1, wherein the forward primer and the reverse primer span a region in the E6 and/or E7 region of the HPV genome that is about 75 nucleotides in length, or less.

24. The method of claim 1, wherein the forward primer and the reverse primer span a region in the E6 and/or E7 region of the HPV genome that is about 97 nucleotides in length.

25. The method of claim 1, wherein the specific amplified PCR product is about 150 nucleotides in length, or less.

26. The method of claim 1, wherein the specific amplified PCR product is about 125 nucleotides in length, or less.

27. The method of claim 1, wherein the specific amplified PCR product is about 100 nucleotides in length, or less.

28. The method of claim 1, wherein the specific amplified PCR product is about 75 nucleotides in length, or less.

29. The method of claim 1, wherein the specific amplified PCR product is about 97 nucleotides in length.

30. The method of claim 1, wherein the PCR reaction is a quantitative PCR reaction, real-time quantitative PCR reaction, digital PCR reaction, or droplet digital PCR (ddPCR) reaction.

31. The method of claim 1, wherein the PCR reaction is a ddPCR reaction.

32. The method of claim 1, comprising contacting the amplified PCR product with a probe that comprises a fluorophore having a fluorescence property that changes upon hybridization.

33. The method of claim 1, comprising contacting the amplified PCR product with a probe that comprises a fluorophore and a quencher.

34. The method of claim 1, wherein the HPV DNA is HPV16 DNA, and the amplified PCR product is contacted with a probe that comprises SEQ ID. NO. 3, 27, 28, 29, 30, 31,32,33,34, or 35.

35. The method of claim 1, wherein the HPV DNA is HPV33 DNA, and the amplified PCR product is contacted with a probe that comprises SEQ ID. NO. 6, 54, 55, 56, 57, 58, 59, 60, 61, or 62.

36. The method of claim 1, wherein the HPV DNA is HPV18 DNA, the amplified PCR product is contacted with a probe that comprises SEQ ID. NO. 83, 84, 85, 86, 87, 88, 89, 90, 91, or 92.

37. The method of any of the preceding claims, further comprising treating the subject with an anticancer therapeutic agent, with radiation therapy, with surgery, or any combination thereof.

38. A composition or kit for detection of HPV16 DNA comprising one or more of the following primer pairs:
(a) a forward primer comprising SEQ ID NO. 1 and a reverse primer comprising SEQ ID NO. 2,
(b) a forward primer comprising SEQ ID NO. 9 and a reverse primer comprising SEQ ID NO. 10,
(c) a forward primer comprising SEQ ID NO. 11 and a reverse primer comprising SEQ ID NO. 12,
(d) a forward primer comprising SEQ ID NO. 13 and a reverse primer comprising SEQ ID NO. 14,
(e) a forward primer comprising SEQ ID NO. 15 and a reverse primer comprising SEQ ID NO. 16,
(f) a forward primer comprising SEQ ID NO. 17 and a reverse primer comprising SEQ ID NO. 18,
(g) a forward primer comprising SEQ ID NO. 19 and a reverse primer comprising SEQ ID NO. 20,
(h) a forward primer comprising SEQ ID NO. 21 and a reverse primer comprising SEQ ID NO. 22,
(i) a forward primer comprising SEQ ID NO. 23 and a reverse primer comprising SEQ ID NO. 24, and
(j) a forward primer comprising SEQ ID NO. 25 and a reverse primer comprising SEQ ID NO. 26.

39. A composition or kit according to claim 38 further comprising a probe that binds to an amplicon spanned by the primer pair.

40. A composition or kit for detection of HPV16 DNA comprising one or more of the following primer pair and probe combinations:
(a) a forward primer comprising SEQ ID NO. 1 (Rama FOR primer) and a reverse primer comprising SEQ ID NO. 2 (Rama REV primer) and a probe comprising SEQ ID NO. 3,
(b) a forward primer comprising SEQ ID NO. 9 (PR HPV16 primer1.FOR) and a reverse primer comprising SEQ ID NO. 10 (PR HPV16 primer1.REV) and a probe comprising SEQ ID NO.27,
(c) a forward primer comprising SEQ ID NO. 11 and a reverse primer comprising SEQ ID NO. 12 and a probe comprising SEQ ID NO. 28,
(d) a forward primer comprising SEQ ID NO. 13 and a reverse primer comprising SEQ ID NO. 14 and a probe comprising SEQ ID NO. 29,
(e) a forward primer comprising SEQ ID NO. 15 and a reverse primer comprising SEQ ID NO. 16 and a probe comprising SEQ ID NO. 30,
(f) a forward primer comprising SEQ ID NO. 17 and a reverse primer comprising SEQ ID NO. 18 and a probe comprising SEQ ID NO. 31,
(g) a forward primer comprising SEQ ID NO. 19 and a reverse primer comprising SEQ ID NO. 20 and a probe comprising SEQ ID NO. 32,
(h) a forward primer comprising SEQ ID NO. 21 and a reverse primer comprising SEQ ID NO. 22 and a probe comprising SEQ ID NO. 33,
(i) a forward primer comprising SEQ ID NO. 23 and a reverse primer comprising SEQ ID NO. 24 and a probe comprising SEQ ID NO. 34,
(j) a forward primer comprising SEQ ID NO. 25 and a reverse primer comprising SEQ ID NO. 26 and a probe comprising SEQ ID NO. 35.

41. A composition or kit according to claim 40 wherein the probe comprises a fluorophore having a fluorescence property that changes upon hybridization.

42. A composition or kit according to any one of claims 38-41 further comprising an HPV16 positive control and/or an HPV16 negative control.

43. A composition or kit for detection of HPV33 DNA comprising one or more of the following primer pairs:
(a) a forward primer comprising SEQ ID NO. 4 and a reverse primer comprising SEQ ID NO. 5,
(b) a forward primer comprising SEQ ID NO. 36 and a reverse primer comprising SEQ ID NO. 37,
(c) a forward primer comprising SEQ ID NO. 38 and a reverse primer comprising SEQ ID NO. 39,
(d) a forward primer comprising SEQ ID NO. 40 and a reverse primer comprising SEQ ID NO. 41,
(e) a forward primer comprising SEQ ID NO. 42 and a reverse primer comprising SEQ ID NO. 43,
(f) a forward primer comprising SEQ ID NO. 44 and a reverse primer comprising SEQ ID NO. 45,
(g) a forward primer comprising SEQ ID NO. 46 and a reverse primer comprising SEQ ID NO. 47,
(h) a forward primer comprising SEQ ID NO. 48 and a reverse primer comprising SEQ ID NO. 49,
(i) a forward primer comprising SEQ ID NO. 50 and a reverse primer comprising SEQ ID NO. 51,
(j) a forward primer comprising SEQ ID NO. 52 and a reverse primer comprising SEQ ID NO. 53.

44. A composition or kit according to claim 43 further comprising a probe that binds to an amplicon spanned by the primer pair.

45. A composition or kit for detection of HPV33 DNA comprising one or more of the following primer pair and probe combinations:
(a) a forward primer comprising SEQ ID NO. 4 and a reverse primer comprising SEQ ID NO. 5 and a probe comprising SEQ ID NO. 6,
(b) a forward primer comprising SEQ ID NO. 36 and a reverse primer comprising SEQ ID NO. 37 and a probe comprising SEQ ID NO. 54,
(c) a forward primer comprising SEQ ID NO. 38 and a reverse primer comprising SEQ ID NO. 39 and a probe comprising SEQ ID NO. 55,
(d) a forward primer comprising SEQ ID NO. 40 and a reverse primer comprising SEQ ID NO. 41 and a probe comprising SEQ ID NO. 56,
(e) a forward primer comprising SEQ ID NO. 42 and a reverse primer comprising SEQ ID NO. 43 and a probe comprising SEQ ID NO. 57,
(f) a forward primer comprising SEQ ID NO. 44 and a reverse primer comprising SEQ ID NO. 45 and a probe comprising SEQ ID NO. 58,
(g) a forward primer comprising SEQ ID NO. 46 and a reverse primer comprising SEQ ID NO. 47 and a probe comprising SEQ ID NO. 59,
(h) a forward primer comprising SEQ ID NO. 48 and a reverse primer comprising SEQ ID NO. 49 and a probe comprising SEQ ID NO. 60,
(i) a forward primer comprising SEQ ID NO. 50 and a reverse primer comprising SEQ ID NO. 51 and a probe comprising SEQ ID NO. 61,
(j) a forward primer comprising SEQ ID NO. 52 and a reverse primer comprising SEQ ID NO. 53 and a probe comprising SEQ ID NO. 62.

46. A composition or kit according to claim 45 wherein the probe comprises a fluorophore having a fluorescence property that changes upon hybridization.

47. A composition or kit according to any one of claims 43-46, further comprising an HPV33 positive control and/or an HPV33 negative control.

48. A composition or kit for detection of HPV18 DNA comprising one or more of the following primer pairs:
(a) a forward primer comprising SEQ ID NO. 63 and a reverse primer comprising SEQ ID NO. 64,
(b) a forward primer comprising SEQ ID NO. 65 and a reverse primer comprising SEQ ID NO. 66,
(c) a forward primer comprising SEQ ID NO. 67 and a reverse primer comprising SEQ ID NO. 68,
(d) a forward primer comprising SEQ ID NO. 69 and a reverse primer comprising SEQ ID NO. 70,
(e) a forward primer comprising SEQ ID NO. 71 and a reverse primer comprising SEQ ID NO. 72,
(f) a forward primer comprising SEQ ID NO. 73 and a reverse primer comprising SEQ ID NO. 74,
(g) a forward primer comprising SEQ ID NO. 75 and a reverse primer comprising SEQ ID NO. 76,
(h) a forward primer comprising SEQ ID NO. 77 and a reverse primer comprising SEQ ID NO. 78,
(i) a forward primer comprising SEQ ID NO. 79 and a reverse primer comprising SEQ ID NO. 80,
(j) a forward primer comprising SEQ ID NO. 81 and a reverse primer comprising SEQ ID NO. 82.

49. A composition or kit according to claim 48, further comprising a probe that binds to an amplicon spanned by the primer pair.

50. A composition or kit for detection of HPV18 DNA comprising one or more of the following primer pair and probe combinations:
(a) a forward primer comprising SEQ ID NO. 63 and a reverse primer comprising SEQ ID NO. 64 and a probe comprising SEQ ID NO. 83,
(b) a forward primer comprising SEQ ID NO. 65 and a reverse primer comprising SEQ ID NO. 66 and a probe comprising SEQ ID NO. 84,
(c) a forward primer comprising SEQ ID NO. 67 and a reverse primer comprising SEQ ID NO. 68 and a probe comprising SEQ ID NO. 85,
(d) a forward primer comprising SEQ ID NO. 69 and a reverse primer comprising SEQ ID NO. 70 and a probe comprising SEQ ID NO. 86,
(e) a forward primer comprising SEQ ID NO. 71 and a reverse primer comprising SEQ ID NO. 72 and a probe comprising SEQ ID NO. 87,
(f) a forward primer comprising SEQ ID NO. 73 and a reverse primer comprising SEQ ID NO. 74 and a probe comprising SEQ ID NO. 88,
(g) a forward primer comprising SEQ ID NO. 75 and a reverse primer comprising SEQ ID NO. 76 and a probe comprising SEQ ID NO. 89,
(h) a forward primer comprising SEQ ID NO. 77 and a reverse primer comprising SEQ ID NO. 78 and a probe comprising SEQ ID NO. 90,
(i) a forward primer comprising SEQ ID NO. 79 and a reverse primer comprising SEQ ID NO. 80 and a probe comprising SEQ ID NO. 91,
(j) a forward primer comprising SEQ ID NO. 81 and a reverse primer comprising SEQ ID NO. 82 and a probe comprising SEQ ID NO. 92.

51. A composition or kit according to claim 50 wherein the probe comprises a fluorophore having a fluorescence property that changes upon hybridization.

52. A composition or kit according to any one of claims 48-51, further comprising an HPV18 control and/or an HPV18 negative control.


SEQUENCE LISTING (92 sequences)

SEQ ID NO: 1 - DNA, 20 bp, Artificial Sequence
  Description of Artificial Sequence Synthetic primer
tatgcacagagctgcaaaca

SEQ ID NO: 2 - DNA, 22 bp, Artificial Sequence
  Description of Artificial Sequence Synthetic primer
gcaaagtcatatacctcacgtc

SEQ ID NO: 3 - DNA, 27 bp, Artificial Sequence
  Description of Artificial Sequence Synthetic probe
tgtgtgtactgcaagcaacagttactg

SEQ ID NO: 4 - DNA, 21 bp, Artificial Sequence
  Description of Artificial Sequence Synthetic primer
ccacagttcgtttatgtgtca

SEQ ID NO: 5 - DNA, 20 bp, Artificial Sequence
  Description of Artificial Sequence Synthetic primer
tgcccataagtagttgctgt

SEQ ID NO: 6 - DNA, 25 bp, Artificial Sequence
  Description of Artificial Sequence Synthetic probe
agtacagcaagtgacctacgaacca

SEQ ID NO: 7 - DNA, 20 bp, Artificial Sequence
  Description of Artificial Sequence Synthetic primer
cggtcgatgtatgtcttgtt

SEQ ID NO: 8 - DNA, 20 bp, Artificial Sequence
  Description of Artificial Sequence Synthetic primer
ctgggtttctctacgtgttc

SEQ ID NO: 9 - DNA, 19 bp, Artificial Sequence
  Description of Artificial Sequence Synthetic primer
tgcaccaaaagagaactgc

SEQ ID NO: 10 - DNA, 20 bp, Artificial Sequence
  Description of Artificial Sequence Synthetic primer
tgtttgcagctctgtgcata

SEQ ID NO: 11 - DNA, 24 bp, Artificial Sequence
  Description of Artificial Sequence Synthetic primer
ggaacaacattagaacagcaatac

SEQ ID NO: 12 - DNA, 21 bp, Artificial Sequence
  Description of Artificial Sequence Synthetic primer
gtccagatgtctttgcttttc

SEQ ID NO: 13 - DNA, 24 bp, Artificial Sequence
  Description of Artificial Sequence Synthetic primer
agcaaagattccataatataaggg

SEQ ID NO: 14 - DNA, 21 bp, Artificial Sequence
  Description of Artificial Sequence Synthetic primer
ttacagctgggtttctctacg

SEQ ID NO: 15 - DNA, 20 bp, Artificial Sequence
  Description of Artificial Sequence Synthetic primer
gcatggagatacacctacat

SEQ ID NO: 16 - DNA, 27 bp, Artificial Sequence
  Description of Artificial Sequence Synthetic primer
ttaattgctcataacagtagagatcag

SEQ ID NO: 17 - DNA, 24 bp, Artificial Sequence
  Description of Artificial Sequence Synthetic primer
acaactgatctctactgttatgag

SEQ ID NO: 18 - DNA, 23 bp, Artificial Sequence
  Description of Artificial Sequence Synthetic primer
ggttacaatattgtaatgggctc

SEQ ID NO: 19 - DNA, 16 bp, Artificial Sequence
  Description of Artificial Sequence Synthetic primer
agaaccggacagagcc

SEQ ID NO: 20 - DNA, 21 bp, Artificial Sequence
  Description of Artificial Sequence Synthetic primer
gtctacgtgtgtgctttgtac

SEQ ID NO: 21 - DNA, 17 bp, Artificial Sequence
  Description of Artificial Sequence Synthetic primer
gactctacgcttcggtt

SEQ ID NO: 22 - DNA, 17 bp, Artificial Sequence
  Description of Artificial Sequence Synthetic primer
gaacagatggggcacac

SEQ ID NO: 23 - DNA, 21 bp, Artificial Sequence
  Description of Artificial Sequence Synthetic primer
tagaatgtgtgtactgcaagc

SEQ ID NO: 24 - DNA, 36 bp, Artificial Sequence
  Description of Artificial Sequence Synthetic primer
cactaattttagaataaaactttaaacatttatcac

SEQ ID NO: 25 - DNA, 36 bp, Artificial Sequence
  Description of Artificial Sequence Synthetic primer
gtgataaatgtttaaagttttattctaaaattagtg

SEQ ID NO: 26 - DNA, 22 bp, Artificial Sequence
  Description of Artificial Sequence Synthetic primer
ggcttttgacagttaatacacc

SEQ ID NO: 27 - DNA, 18 bp, Artificial Sequence
  Description of Artificial Sequence Synthetic probe
aggacccacaggagcgac

SEQ ID NO: 28 - DNA, 29 bp, Artificial Sequence
  Description of Artificial Sequence Synthetic probe
attaggtgtattaactgtcaaaagccact

SEQ ID NO: 29 - DNA, 25 bp, Artificial Sequence
  Description of Artificial Sequence Synthetic probe
tggaccggtcgatgtatgtcttgtt

SEQ ID NO: 30 - DNA, 32 bp, Artificial Sequence
  Description of Artificial Sequence Synthetic probe
tgcatgaatatatgttagatttgcaaccagag

SEQ ID NO: 31 - DNA, 25 bp, Artificial Sequence
  Description of Artificial Sequence Synthetic probe
aaatagatggtccagctggacaagc

SEQ ID NO: 32 - DNA, 24 bp, Artificial Sequence
  Description of Artificial Sequence Synthetic probe
ttgcaagtgtgactctacgcttcg

SEQ ID NO: 33 - DNA, 25 bp, Artificial Sequence
  Description of Artificial Sequence Synthetic probe
ttggaagacctgttaatgggcacac

SEQ ID NO: 34 - DNA, 34 bp, Artificial Sequence
  Description of Artificial Sequence Synthetic probe
ttttcgggatttatgcatagtatatagagatggg

SEQ ID NO: 35 - DNA, 36 bp, Artificial Sequence
  Description of Artificial Sequence Synthetic probe
tgtatggaacaacattagaacagcaatacaacaaac

SEQ ID NO: 36 - DNA, 23 bp, Artificial Sequence
  Description of Artificial Sequence Synthetic primer
atgtttcaagacactgaggaaaa

SEQ ID NO: 37 - DNA, 18 bp, Artificial Sequence
  Description of Artificial Sequence Synthetic primer
ccacgcactgtagttcaa

SEQ ID NO: 38 - DNA, 23 bp, Artificial Sequence
  Description of Artificial Sequence Synthetic primer
ggagacaactatacacaacattg

SEQ ID NO: 39 - DNA, 28 bp, Artificial Sequence
  Description of Artificial Sequence Synthetic primer
ctatatacaactgttaaatctgcaaatg

SEQ ID NO: 40 - DNA, 29 bp, Artificial Sequence
  Description of Artificial Sequence Synthetic primer
gcatttgcagatttaacagttgtatatag

SEQ ID NO: 41 - DNA, 30 bp, Artificial Sequence
  Description of Artificial Sequence Synthetic primer
ctatattcactaattttagataagaaccgc

SEQ ID NO: 42 - DNA, 32 bp, Artificial Sequence
  Description of Artificial Sequence Synthetic primer
aaacctttaaatgaaatattaattaggtgtat

SEQ ID NO: 43 - DNA, 21 bp, Artificial Sequence
  Description of Artificial Sequence Synthetic primer
ccaacgacccgaaatattatg

SEQ ID NO: 44 - DNA, 21 bp, Artificial Sequence
  Description of Artificial Sequence Synthetic primer
cataatatttcgggtcgttgg

SEQ ID NO: 45 - DNA, 16 bp, Artificial Sequence
  Description of Artificial Sequence Synthetic primer
gtgtcctctcatggcg

SEQ ID NO: 46 - DNA, 21 bp, Artificial Sequence
  Description of Artificial Sequence Synthetic primer
agccaacgttaaaggaatatg

SEQ ID NO: 47 - DNA, 20 bp, Artificial Sequence
  Description of Artificial Sequence Synthetic primer
ctgagctgtcacttaattgc

SEQ ID NO: 48 - DNA, 21 bp, Artificial Sequence
  Description of Artificial Sequence Synthetic primer
tgagcaattaagtgacagctc

SEQ ID NO: 49 - DNA, 15 bp, Artificial Sequence
  Description of Artificial Sequence Synthetic primer
gctgtggctggttgt

SEQ ID NO: 50 - DNA, 18 bp, Artificial Sequence
  Description of Artificial Sequence Synthetic primer
gatggacaagcacaacca

SEQ ID NO: 51 - DNA, 21 bp, Artificial Sequence
  Description of Artificial Sequence Synthetic primer
tatggttcgtaggtcacttgc

SEQ ID NO: 52 - DNA, 22 bp, Artificial Sequence
  Description of Artificial Sequence Synthetic primer
gtaacaccacagttcgtttatg

SEQ ID NO: 53 - DNA, 20 bp, Artificial Sequence
  Description of Artificial Sequence Synthetic primer
actgtgcccataagtagttg

SEQ ID NO: 54 - DNA, 23 bp, Artificial Sequence
  Description of Artificial Sequence Synthetic probe
acattgcatgatttgtgccaagc

SEQ ID NO: 55 - DNA, 29 bp, Artificial Sequence
  Description of Artificial Sequence Synthetic probe
cctttgcaacgatctgaggtatatgattt

SEQ ID NO: 56 - DNA, 31 bp, Artificial Sequence
  Description of Artificial Sequence Synthetic probe
agggaaatccatttggaatatgtaaactgtg

SEQ ID NO: 57 - DNA, 33 bp, Artificial Sequence
  Description of Artificial Sequence Synthetic probe
agaaaaaaaacgacatgtggatttaaacaaacg

SEQ ID NO: 58 - DNA, 23 bp, Artificial Sequence
  Description of Artificial Sequence Synthetic probe
cgacgtagagaaactgcactgtg

SEQ ID NO: 59 - DNA, 34 bp, Artificial Sequence
  Description of Artificial Sequence Synthetic probe
agatttatatcctgaaccaactgacctatactgc

SEQ ID NO: 60 - DNA, 21 bp, Artificial Sequence
  Description of Artificial Sequence Synthetic probe
tgaggatgaaggcttggaccg

SEQ ID NO: 61 - DNA, 26 bp, Artificial Sequence
  Description of Artificial Sequence Synthetic probe
ccacagttcgtttatgtgtcaacagt

SEQ ID NO: 62 - DNA, 24 bp, Artificial Sequence
  Description of Artificial Sequence Synthetic probe
gcaagtgacctacgaaccatacag

SEQ ID NO: 63 - DNA, 14 bp, Artificial Sequence
  Description of Artificial Sequence Synthetic primer
atggcgcgctttga

SEQ ID NO: 64 - DNA, 22 bp, Artificial Sequence
  Description of Artificial Sequence Synthetic primer
ctatgtcttgcagtgaagtgtt

SEQ ID NO: 65 - DNA, 17 bp, Artificial Sequence
  Description of Artificial Sequence Synthetic primer
tctgtgcacggaactga

SEQ ID NO: 66 - DNA, 23 bp, Artificial Sequence
  Description of Artificial Sequence Synthetic primer
ctgtaagttccaatactgtcttg

SEQ ID NO: 67 - DNA, 24 bp, Artificial Sequence
  Description of Artificial Sequence Synthetic primer
gtgtatattgcaagacagtattgg

SEQ ID NO: 68 - DNA, 36 bp, Artificial Sequence
  Description of Artificial Sequence Synthetic primer
ctctaattctagaataaaaatctatacatttatggc

SEQ ID NO: 69 - DNA, 24 bp, Artificial Sequence
  Description of Artificial Sequence Synthetic primer
attattcagactctgtgtatggag

SEQ ID NO: 70 - DNA, 21 bp, Artificial Sequence
  Description of Artificial Sequence Synthetic primer
gtgtctaagtttttctgctgg

SEQ ID NO: 71 - DNA, 15 bp, Artificial Sequence
  Description of Artificial Sequence Synthetic primer
gcggtgccagaaacc

SEQ ID NO: 72 - DNA, 17 bp, Artificial Sequence
  Description of Artificial Sequence Synthetic primer
ggttgcagcacgaatgg

SEQ ID NO: 73 - DNA, 23 bp, Artificial Sequence
  Description of Artificial Sequence Synthetic primer
gacattgtattgcatttagagcc

SEQ ID NO: 74 - DNA, 24 bp, Artificial Sequence
  Description of Artificial Sequence Synthetic primer
aactccatctatttcatcgttttc

SEQ ID NO: 75 - DNA, 22 bp, Artificial Sequence
  Description of Artificial Sequence Synthetic primer
caattaagcgactcagaggaag

SEQ ID NO: 76 - DNA, 18 bp, Artificial Sequence
  Description of Artificial Sequence Synthetic primer
cattgtgtgacgttgtgg

SEQ ID NO: 77 - DNA, 18 bp, Artificial Sequence
  Description of Artificial Sequence Synthetic primer
accacaacgtcacacaat

SEQ ID NO: 78 - DNA, 22 bp, Artificial Sequence
  Description of Artificial Sequence Synthetic primer
tgctgagctttctactactagc

SEQ ID NO: 79 - DNA, 21 bp, Artificial Sequence
  Description of Artificial Sequence Synthetic primer
gctagtagtagaaagctcagc

SEQ ID NO: 80 - DNA, 15 bp, Artificial Sequence
  Description of Artificial Sequence Synthetic primer
acaccacggacacac

SEQ ID NO: 81 - DNA, 15 bp, Artificial Sequence
  Description of Artificial Sequence Synthetic primer
ggcgaccctacaagc

SEQ ID NO: 82 - DNA, 21 bp, Artificial Sequence
  Description of Artificial Sequence Synthetic primer
ctgtcttgcaatatacacagg

SEQ ID NO: 83 - DNA, 19 bp, Artificial Sequence
  Description of Artificial Sequence Synthetic probe
tccaacacggcgaccctac

SEQ ID NO: 84 - DNA, 30 bp, Artificial Sequence
  Description of Artificial Sequence Synthetic probe
cacttcactgcaagacatagaaataacctg

SEQ ID NO: 85 - DNA, 27 bp, Artificial Sequence
  Description of Artificial Sequence Synthetic probe
tatagagacagtataccgcatgctgca

SEQ ID NO: 86 - DNA, 33 bp, Artificial Sequence
  Description of Artificial Sequence Synthetic probe
ctgggttatacaatttattaataaggtgcctgc

SEQ ID NO: 87 - DNA, 21 bp, Artificial Sequence
  Description of Artificial Sequence Synthetic probe
ctgggcactatagaggccagt

SEQ ID NO: 88 - DNA, 23 bp, Artificial Sequence
  Description of Artificial Sequence Synthetic probe
cgagcaattaagcgactcagagg

SEQ ID NO: 89 - DNA, 38 bp, Artificial Sequence
  Description of Artificial Sequence Synthetic probe
agaaaacgatgaaatagatggagttaatcatcaacatt

SEQ ID NO: 90 - DNA, 28 bp, Artificial Sequence
  Description of Artificial Sequence Synthetic probe
tgtatgtgttgtaagtgtgaagccagaa

SEQ ID NO: 91 - DNA, 20 bp, Artificial Sequence
  Description of Artificial Sequence Synthetic probe
cgaccttcgagcattccagc

SEQ ID NO: 92 - DNA, 22 bp, Artificial Sequence
  Description of Artificial Sequence Synthetic probe
ctgtgcacggaactgaacactt